(19) United States
(12) Patent Application Publication
(10) Pub. No.: US 2011/0229496 A1
(43) Pub. Date: Sep. 22, 2011

(54) METHOD FOR REGULATING SURVIVAL AND MEMORY OF T HELPER 1 CELLS

(75) Inventor: Linda M. Bradley, La Jolla, CA (US)

(73) Assignee: Sanford-Burnham Medical Research Institute, La Jolla, CA (US)

(21) Appl. No.: 13/051,922

(22) Filed: Mar. 18, 2011

Related U.S. Application Data
(60) Provisional application No. 61/315,798, filed on Mar. 19, 2010.

(51) Int. Cl. A61K 39/395; A61K 35/55; A61P 3/10; A61P 11/00; A61P 7/06; A61P 19/02; A61P 17/06; A61P 31/16; C12N 5/078; C12Q 1/02; C40B 30/06; A61P 29/00; A61P 17/00; A61P 25/00

(52) U.S. Cl. 424/173.1; 424/93.7; 435/375; 435/29; 506/10

(57) ABSTRACT

The present invention provides a method of inhibiting survival of T helper 1 (Th1) memory cells by contacting or administering a population of Th1 cells with an agent that inhibits CD44 receptor expression or activation. The invention further provides a method of stimulating memory Th1 cell survival comprising contacting or administering Th1 cells with an agent that increases CD44 receptor activation or expression in the cells. The invention additionally provides a method of screening for agents capable of modulating Th1 cell survival.

### CROSS REFERENCE TO RELATED APPLICATION

This application claimed the benefit of priority under 35 U.S.C. §119(e) of U.S. Ser. No. 61/315,798 filed Mar. 19, 2010, the entire content of which is incorporated herein by reference.

### GRANT INFORMATION

This invention was made with government support under NIH Grant Nos. AI061615 and AI046530 awarded by the National Institutes of Health. The government has certain rights in the invention.

### BACKGROUND OF THE INVENTION

1. Field of the Invention

The invention relates generally to immunology and more specifically to regulation of survival and memory of T helper 1 (Th1) cells.

2. Background Information

In becoming memory cells, T cells undergo stages of dramatic expansion and contraction that depend upon regulated cell death and thereafter are maintained by survival signals from the environment. Survival of T cells during a response can be profoundly affected by the availability of costimulatory molecules and cytokines that modulate engagement of death pathways. Once a response subsides, common gamma chain cytokines, such as interleukin-7 (IL-7) and IL-15, are essential to the homeostatic control of T cell memory. However, as predominantly mobile populations, both effector and memory T cells have the potential to receive additional signals through adhesive interactions with the extracellular matrix (ECM) or other cells.

CD44 is an adhesion molecule that is expressed by most cells and mediates binding to the ECM and other cells via its only known in vivo ligand, the glycosaminoglycan hyaluronic acid (HA). CD44 expression is upregulated on naive T cells after activation via the T cell receptor (TCR) and high expression is maintained indefinitely on memory cells. As a consequence, elevated expression of CD44 is generally used to identify antigen-experienced T cells. CD44 is associated with cell migration and together with HA has been implicated in numerous biological processes that are regulated by migrating cells. The function of CD44 differs for different cell types and additional roles in the regulation of proliferation and apoptosis have been described.

CD44 is the product of a single gene that gives rise to a family of HA-binding molecules by alternative exon RNA splicing. In addition to the nonvariant or standard form of CD44, at least five isoforms are generated through translation of various combinations of 10 variable exons, which are inserted into a single site in the membrane proximal region of the extracellular domain. Additional cell type-specific posttranslational modifications of CD44 include differences in glycosylation. The variable forms of CD44 contribute to functional variations that allow for diverse interactions of cells with their environments through a variety of signaling events, which are not yet fully defined and can vary in different cell types.

Whereas CD44 has the potential to participate in several processes associated with immune responses, the physiological functions of CD44 in T cells in vivo remain ill defined. It has been established that T cells bind HA, and that either HA binding or TCR signaling can augment the adhesive function and expression of CD44. CD44 together with VLA-4 (α4 integrin) can regulate T cell migration into sites of inflammation, and the association of these receptors correlates with enhanced T cell motility and survival after TCR stimulation in vitro. The binding of CD44 expressed on T cells to HA on the surface of dendritic cells (DCs) can promote cell clustering that can be blocked by HA inhibitors. Although ligation of CD44 does not elicit proliferation of T cells, it can activate the TCR signaling-associated src family kinases Lck and Fyn. This suggests that induction of signaling events by CD44 impacts the T cell response, including that to TCR engagement. CD44 has been associated with both resistance and susceptibility of activated T cells to apoptosis, suggesting that it participates in the control of expansion. However, although CD44 is broadly connected with the regulation of T cell responses, distinguishing direct roles in vivo has remained elusive, prompting study of its function in CD4+ T cells.

### SUMMARY OF THE INVENTION

The present invention is based on the seminal discovery that CD44 expression and engagement are important for the generation of memory Th1 cells by promoting effector cell survival. Additionally, Th1 cell survival may be decreased by inhibiting CD44 receptor expression or ligand binding.

Accordingly, in one embodiment, the present invention provides a method of inhibiting survival of Th1 memory cells. The method includes contacting or administering a population of Th1 cells with an agent that inhibits CD44 receptor expression or engagement, thereby inhibiting survival of the Th1 cells. In various aspects, the agent may be an agent that inhibits engagement of the CD44 receptor, such as a CD44 receptor antagonist, or the agent may inhibit CD44 receptor expression, such as a nucleic acid molecule. In various aspects, the contacting or administering may be in vivo or in vitro. In one aspect where the contacting or administering is performed in vivo, the contacting or administering is performed in a subject having an immune system disorder associated with expression or signaling of CD44 receptors. In an exemplary aspect, the disorder is diabetes mellitus type 1.

In another embodiment, the invention provides a method of stimulating Th1 memory cell survival. The method includes contacting or administering Th1 cells with an agent that increases CD44 receptor engagement in the cells, thereby stimulating memory T helper 1 (Th1) cell survival. In various aspects, the agent is a CD44 receptor agonist. In various aspects, the contacting or administering may be in vivo or in vitro. In one aspect where the contacting or administering is performed in vivo, the contacting or administering is performed in a subject having or at risk of having an infection with a bacterial or viral agent. In an exemplary aspect, the viral agent is influenza.

In another embodiment, the present invention provides a method of screening for an agent that modulates Th1 memory cell survival. The method includes contacting or administering a test agent with a Th1 cell or population thereof, and detecting an increase or decrease in the CD44 receptor expression or signaling as compared with expression or signaling prior to contacting or administering the agent, thereby identifying the test agent as an agent that modulates Th1 cell survival. In one aspect, the test agent is an agent that increases CD44 receptor expression or signaling, such as a CD44 receptor agonist. In another aspect, the test agent is an agent that decreases CD44 receptor expression or signaling, such as a CD44 receptor antagonist or antisense nucleic acid molecule. In various aspects, the Th1 cells may be contacted in vivo or in vitro. In one aspect, the screening is performed via a high throughput format.

### BRIEF DESCRIPTION OF THE DRAWINGS

FIG. 1 shows potential mechanisms by which CD44 can modulate Th1 cell death. FIG. 1A shows that CD44 may inhibit apoptosis by sequestering FAS and thereby preventing assembly of death-inducing signaling complex (DISC). Without DISC formation, Fas ligand (FasL) cannot engage FAS, which precludes downstream activation of caspases that would lead to apoptosis. FIG. 1B shows that ligation of CD44 (e.g., through HA) can facilitate aggregation of CD44-integrin-kinase signaling components in lipid rafts. Src family kinases, such as Lck, associate with the cytoplasmic tail of CD44 and activate PI3L/Akt signaling. Alternatively, binding of either CD44 or VLA-4, which form a heterodimer at the cell surface, could activate PI3K via FAK. Activation of PI3K/Akt is associated with cell survival. Akt can inhibit Fas-mediated CD4 T cell death by interfering with DISC assembly. In addition, the mTOR pathway may be engaged to support the survival and expansion of T cells.

FIG. 2 shows multiple graphical representations. FIG. 2A is a graph of T cell recovery. FIG. 2B is a graph depicting T cell percentage in the Vβ5⁺, CD4⁺ population taken from bronchoalveolar lavage (BAL), lung, peripheral lymph nodes (PLN), spleen and mediastinal lymph nodes (MSLN).

FIG. 3 shows multiple graphical representations. FIG. 3A shows histograms depicting T cell division via CFSE analysis. FIG. 3B shows histograms depicting T cell recoveries of Tg⁺ donor cells. FIG. 3C shows histograms depicting IFN-γ and TNF-α production by wild type (WT) and CD44^{-/-} cells. FIG. 3D shows a histogram depicting recovery of divided T cells. FIG. 3E shows a graph depicting recovery of Tg⁺ cells (mean±SEM, n=3-4/group).

FIG. 4 shows multiple graphical representations. FIG. 4A shows histograms depicting Tg⁺ cell division. FIG. 4B shows histograms depicting Tg⁺ cell recoveries. FIG. 4C shows histograms depicting Tg⁺ donor cell division. FIG. 4D shows histograms depicting recovery of donor CD4⁺ T cells that had undergone one or more divisions in the MSLN and spleen 8 and 13 days after infection. FIG. 4E shows histograms depicting recovery of BrdU⁺ CD4⁺ T cells. FIG. 4F shows a histogram depicting percentages of CD154⁺IFN-γ⁺ virus specific CD4⁺ T cells. For FIGS. 4B, D-F, mean±SEM, n=3-4/group.

FIG. 5 shows multiple graphical representations for apoptosis of responding CD44−/−CD4 cells. C57BL/6 mice were given WT and CD44−/− OT-II cells (1.5×10⁶ each) and infected with WSN-OVA_{II}. FIG. 5A shows histograms depicting T cell numbers assessed by binding of Annexin V and exclusion of 7AAD by WT Tg⁺ cells and CD44^{-/-} Tg⁺ cells. Apoptosis is assessed by binding of Annexin V and exclusion of 7AAD by WT Tg+ cells (shaded histograms) and CD44−/− Tg+ (open histograms) cells after gating on Thy1.1+ or Ly5.1+ cells in the Vβ5+, CD4+ population. FIG. 5B shows graphs depicting viable recoveries of WT and CD44^{-/-} donor Tg⁺ cells in the MSLN and lungs (mean±SEM, n=5/group). Viable donor cell recovery for WT cells and CD44−/− cells is shown. Mean±SEM, n=5/group. FIG. 5C shows a histogram depicting caspase 8 activation in T cells. Caspase 8 activation as assessed using a fluorophore-modified substrate with dispersed MSLN cells from recipients of WT and CD44−/− CD4 cells on day 7 after influenza virus infection. The fluorescence induced by activated caspase 8 for WT Tg+ cells (shaded histogram) and CD44−/− Tg+ cells (open histogram) are shown in 7AAD−, Thy1.1+ or Ly5.1+ cells in the Vβ5+, CD4+ population. The results are representative of 6 recipients.

FIG. 6 shows multiple graphical representations. FIG. 6A shows graphs depicting frequency of Tg⁺ cells, gated on the Vβ5⁺, CD4⁺ population in the lungs and spleen at the indicated times (mean±SEM, n=4/group). FIG. 6B shows a graph depicting recovery of donor cells from the spleen (mean±SEM, n=4/group). FIG. 6C shows graphs depicting frequencies of donor cells recovered at various time points.

FIG. 7 shows multiple graphical representations. FIG. 7A shows a graph depicting T cell numbers recovered from the spleen and lungs of C57BL/6 mice. FIG. 7B shows histograms depicting donor CD4⁺ T cells recovered from the MSLN and spleen of C57BL/6 mice. FIG. 7C shows histograms depicting donor CD4⁺ T cells recovered from the MSLN and spleen of C57BL/6 m. For FIGS. 7A-C, mean±SEM, n=3-4/group. FIG. 7D shows a graph depicting relative densitometry of phospho Akt on immunoblot data.

FIG. 8 shows that Anti-CD44 treatment in vivo does not deplete adaptive T regulatory cells (aTregs or aTreg cells) or naturally occurring regulatory T cells (nTregs or nTreg cells). FIG. 8A shows that aTregs are generated in vitro from BDC 2.5 Thy1.1 CD25− CD4+ T cells and injected i.v. into 8 week old NOD mice (2×10⁶/recipient. n=3/group). At the time of cell transfer, the mice are given either rat IgG or anti-CD44 (IM7) i.p. in a dose of 300 μg/recipient. Thereafter mAb treatment is 2×/week for 14 days. The indicated tissues are analyzed for the presence of donor cells at day 14 (PLN=pancreatic LN, LN=pooled superficial LN). FIG. 8B shows that NOD FoxP3-GFP knock in reporter mice are given rat IgG or anti-CD44 for 2 weeks as in FIG. 8A. The frequency of CD4+ nTregs is determined by FoxP3+ GFP expression. None of the tissues show statistical significance between the control and anti-CD44 treatment in either FIG. 8A or 8B.

FIG. 9 shows that anti-CD44 depletes IFN-γ+ CD4 cells. FIG. 9A shows that NOD IFN-γ reporter mice are given 300 μg anti-CD44 (IM7) or control Ig 2× (days 0 and 4). On day 7, cells from the indicated tissues were stained for CD4 and CD44. The plots are gated on CD4+ cells. FIG. 9B shows that OT-II Thy1.1 CD4 cells are activated for 4 days with anti-CD3/CD28 and treated at 10⁶/ml with 25 μg/ml of the following antibodies: rat IgG, the anti-CD44 mAbs IM7, KM201, IRAWB 14, or with anti-Thy 1.1 (F7D5) as a control, followed by incubation with rabbit complement. Viable cell recovery was determined by trypan blue uptake.

FIG. 10 shows that polyclonal aTreg cells that reverse Type 1 diabetes (T1D) persist as CD25− memory cells. FIG. 10A shows that polyclonal (NOD.Ly5.2) aTreg cells are injected into spontaneously diabetic 8 wk old NOD mice (Ly5.1) in a dose of 2×10⁶/recipient) (n=8) at 1 wk following a blood glucose reading >250 mg/dl. Blood glucose levels are monitored weekly thereafter. Maintenance of FoxP3+ phenotype in the pancreas in vivo in mice injected at 3 months of age with diabetogenic splenic T cells, and treated with aTreg cells 6 weeks later at the time of diabetes onset. Shown are cells from the pancreata pooled from two recipients at 6 months of age. FIG. 10B shows Thy1.1+, donor aTreg cells in the lymph nodes at 2 years after diabetes reversal (left), and FoxP3 expression by the donor population (right).

FIG. 11 shows IL-7Rα expression with the development of memory aTregs. FIG. 11A shows IL-7Rα and CD62L expression at 9 months after aTreg cell transfer and diabetes reversal. FIG. 11B shows that aTreg cells rested in the absence of rIL-7 (open histograms) are compared to aTreg cells right after differentiation (shaded histograms) for expression of IL-7Rα and CD25. FIG. 11C shows that the rested cells are restimulated with PMA/Ionomycin and stained for intracellular TGFβ and IL-10. Shown are histograms of CD4+ gated T cells; the stained cells (shaded) are compared to isotype controls (open).

FIG. 12 shows that aTregs are phenotypically stable after lymphopenia driven expansion. FIG. 12A shows aTreg cells that are differentiated and rested as in FIG. 10 then CFSE-labeled and transferred in a dose of 2×10⁶ cells into NOD.Scid mice. Eleven days later, the cells are tested for proliferation by CFSE dilution in comparison to naive CD4+ T cells. Representative histograms are gated on CD4+ Thy1.1+ donor cells. FIG. 12B shows the recovery of the donor cells from the spleens (n=4 for naive, n=5 for rested aTreg). FIG. 12C shows expression of IL-7Rα and CD62L. FIG. 12D shows FoxP3 and CD25 after gating on the cells shown in FIG. 12B.

FIG. 13 shows that aTreg cells are IL-7-dependent for survival. FIG. 13A shows that polyclonal aTreg cells are induced from CD4+CD25− cells from FoxP3-GFP reporter mice. The cells are injected in a dose of 3×10⁶ into NOD.Scid recipients and treated with IgG or anti-IL-7 neutralizing monoclonal antibody (mAb) at the time of injection and on days 3 and 7, 300 μg/injection. On day 10, the cells from the spleen are analyzed for GFP+ cells (n=5/group). FIG. 13B shows that aTreg cells are induced from CD4+CD25− cells from B6Ly5.1 mice. The cells are transferred in a dose of 3×10⁶ into either IL-7R or IL-7−/− recipients (B6Ly5.2). The mice are evaluated on day 14. The FoxP3+ donor cells (left) are enumerated, and IL-7Rα expression (left) was analyzed by FACS after gating on FoxP3+ donor cells from the spleen on day 14 (n=5/group).

FIG. 14 shows B cells as APC for aTregs. FIG. 14A shows that aTregs are generated from BDC2.5 CD4 cells with anti-CD3 stimulation, or with purified B cells and mimetope peptide. The cultures are supplemented with rIL-2, TGF-β and anti-IFN-γ. The number of FoxP3+ CD4 T cells is quantitated. FIG. 14B shows that polyclonal aTreg cells are cultured in various doses with 10⁴ splenic APC that are precultured for 1 day with 3 μg/ml islet proteins, LN proteins, or no Ag. After 48 hours the culture supernatants are tested for IL-10 secretion by cytokine bead arrays (Luminex).

FIG. 15 shows that aTreg cells require TGF-β for a response in the pancreas and protection from T1D. FIG. 15A shows that aTreg cells generated from BDC 2.5 Thy1.1 CD4+ CD25− T cells are stained for FoxP3 before (left) and after resting for 3 days (middle) or for 1 week in the presence of rIL-7 and anti-TGF-β (right). The plots are gated on CD4+ cells; stained cells (shaded) isotype controls (open). FIGS. 15B-15D show that aTregs are induced from BDC 2.5 Thy1.1 CD4+ CD25− T and injected into NOD mice (2×10⁶, n=4/group). The recipients are given 300 μg control IgG or anti-TGF-β and then treated every 3-4 days. FIG. 15B shows recovery of donor cells from the pooled pancreata. FIG. 15C shows FoxP3 expression by Thy1.1+ gated cells from the spleen of a representative anti-TGF-β treated animal. FIG. 15D shows diabetes incidence after treatment of NOD mice with diabetogenic spleen cells (4×10⁶/recipient) without aTreg cells, or after injection of aTreg cells (2×10⁶/receipt). The mice are given control IgG or anti-TGF-β for 4 weeks (n=5 group).

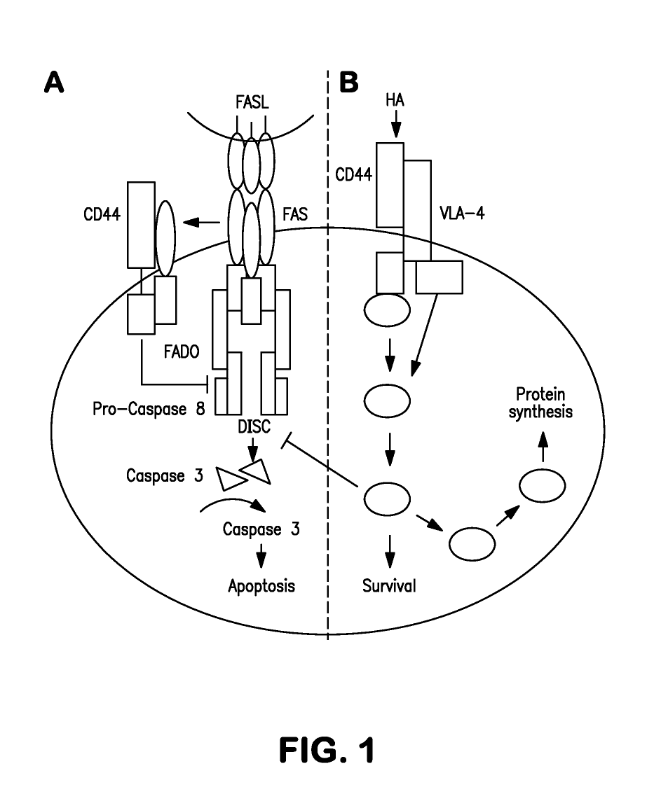

### DETAILED DESCRIPTION OF THE INVENTION

The present invention is based on the seminal discovery that CD44 expression and stimulation is important for the generation of memory Th1 cells by promoting effector cell survival. Additionally, Th1 cell survival may be decreased by inhibiting CD44 receptor expression or activation.

Optimal immunity to microorganisms depends upon the regulated death of clonally expanded effector cells and the survival of a cohort of cells that become memory cells. After activation of naive T cells, CD44, a widely expressed receptor for extracellular matrix components, is upregulated. High expression of CD44 remains on memory cells and despite its wide usage as a “memory marker,” its function is unknown. It has been discovered that CD44 is important for the generation of memory T helper 1 (Th1) cells by promoting effector cell survival. This dependency was not found in Th2, Th17, or CD8⁺ T cells despite similar expression of CD44 and the absence of splice variants in all subsets. CD44 limited Fas-mediated death in Th1 cells and its ligation engaged the phosphoinositide 3-kinase-Akt kinase signaling pathway that regulates cell survival. The difference in CD44-regulated apoptosis resistance in T cell subpopulations has important implications in a broad spectrum of diseases.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

In general, reference to a T helper 1 cell or Th1 cell is intended to refer to a single cell, while reference to Th1 cells is intended to refer to more than one cell. However, one of skill in the art would understand that reference to Th1 cells is intended to include a population of Th1 cells including one or more Th1 cells.

As used herein, the term “sample” refers to any sample suitable for the methods provided by the present invention. The sample may be any sample that includes Th1 cells or fraction thereof and/or that may be used such that CD44 receptor activation, signaling or expression may be detected in association with Th1 cells. In one aspect, the sample is a biological sample, including, for example, a bodily fluid, an extract from a cell, which can be a crude extract or a fractionated extract, a chromosome, an organelle, or a cell membrane; a cell; genomic DNA, RNA, or cDNA, which can be in solution or bound to a solid support; a tissue; or a sample of an organ. A biological sample, for example, from a human subject, can be obtained using well known and routine clinical methods (e.g., a biopsy procedure). Sources of samples and/or Th1 cells may include whole blood, organs and tissue, such as lymphatic tissue including the lymph nodes (e.g., peripheral and mediastinal) spleen, tonsils, adenoids and the thymus, bronchoalveolar lavage (BAL) and the like.

By using a murine model of influenza virus infection in which a Th1 cell response is induced, it was discovered that memory in CD4⁺ T cells failed to develop in the absence of CD44 engagement. Although the development of effectors appeared to proceed normally without CD44, CD4⁺ T cells failed to survive because of apoptosis that engaged caspase-8, suggesting the involvement of extrinsic death-receptor signaling. Unexpectedly, Th1 cells, but not Th2, Th17, or activated CD8⁺ T cells, showed a CD44 requirement for survival and resistance to apoptosis induced by Fas-engagement in vitro, which correlated with higher expression of Fas but not with differences in CD44. Further, ligation of CD44 in vivo enhanced Th1 cell accumulation and in vitro engaged the Akt kinase signaling pathway, which can promote survival of activated CD4+ T cells. Thus, CD44 maintained Th1 cells through active control, which could be mediated by physical contacts with HA in the ECM or on other cells. The results supported the concept that other subsets of T cells were less susceptible to death in part because of inherent differences in Fas expression. This differential regulation may permit strategies for immunotherapeutic targeting of Th1 cells in pathological responses to infections and in autoimmune diseases.

Activated Th1 cells stimulate strong cellular immunity. In general, Th1 responses are stimulated by intracellular pathogens (e.g., viruses, some mycobacteria, yeasts, and parasitic protozoans). The cells produce a number of cytokines known as Th1 cytokines or Type 1 cytokines and including IL2, IFN-gamma, IL12 and TNF-alpha which are important in eliciting an immune response to battle infection.

Accordingly, in one embodiment, the invention provides a method of stimulating Th1 memory cell survival. The method includes contacting or administering Th1 cells with an agent that increases CD44 receptor engagement and signaling in the cells, thereby stimulating Th1 cell survival. The Th1 cells may be contacted in vivo or in vitro. In an exemplary aspect, contacting or administering is performed in a subject having or at risk of having an infection, such as by a bacterial or viral agent, for example, influenza. In this respect, it is expected that an increased CD44 response would enhance immunity of the subject, thereby aiding the subject in preventing or overcoming infection.

As used herein, receptor engagement is intended to include activation of the receptor, via ligand binding for example, as well as down stream signaling events triggered by receptor binding. For example, activation or engagement of CD44 limited Fas-mediated death in Th1 cells and its ligation engaged the phosphoinositide 3-kinase-Akt kinase signaling pathway that regulates cell survival. The difference in CD44-regulated apoptosis resistance in T cell subpopulations has important implications in a broad spectrum of diseases.

The term “subject” as used herein refers to any individual or patient to which the methods of the present invention may be performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

The dependence of Th1 cell survival on CD44 expression and engagement implicates CD44 as a key target in inhibiting Th1 cell survival leading to Th1 cell death. This phenomenon is expected to be useful in treatment of autoimmune diseases arising from overactive immune responses of the body against substances and tissues normally present in the body. Accordingly, in another embodiment, the present invention provides a method of inhibiting survival of Th1 memory cells. The method includes contacting or administering a population of Th1 cells with an agent that inhibits CD44 receptor expression or engagement, thereby inhibiting survival of the Th1 cells. In various aspects, the contacting or administering may be in vivo or in vitro.

Targeting CD44 to inhibit expression and receptor engagement, thereby decreasing cell survival by increasing apoptosis and cell death may be especially significant in treatment of autoimmune diseases. As such, in various aspects, the contacting or administering is performed in a subject in vivo, wherein the subject has, or is at risk of having an autoimmune disorder. As used herein, autoimmune disorders include ankylosing spondylitis, Chagas disease, chronic obstructive pulmonary disease, Crohns disease, dermatomyositis, diabetes mellitus type 1, endometriosis, Goodpasture's syndrome, Graves' disease, Guillain-Barré syndrome (GBS), Hashimoto's disease, hidradenitis suppurativa, Kawasaki disease, IgA nephropathy, idiopathic thrombocytopenic purpura, interstitial cystitis, Lupus erythematosus, mixed connective tissue disease, morphea, myasthenia gravis, narcolepsy, neuromyotonia, pemphigus vulgaris, pernicious anaemia, psoriasis, psoriatic arthritis, polymyositis, primary biliary cirrhosis, rheumatoid arthritis, scleroderma, Sjögren's syndrome, stiff person syndrome, temporal arteritis, ulcerative colitis, vasculitis, vitiligo, and Wegener's granulomatosis.

One of skill in the art would understand that in vivo contacting or administering of a subject with an agent that inhibits signaling by CD44 or expression thereof, may further include treating or contacting or administering the subject with transplant cells of appropriate types to facilitate treatment. For example, in the treatment of a subject suffering from diabetes mellitus type 1 the method may further include treating the subject with an islet cell transplantation.

One of skill in the art would appreciate that a number of methods for generating and delivering transplants to a subject are known and suitable for use in the present invention. For example, transplants may be generated ex vivo, such as by two or three dimensional culturing of cell to generate a tissue suitable for transplant. Similarly, cells may be implanted and cultured in vivo and thus the transplant is generated in vivo.

One of skill in the art would also appreciate that a number of different cell types may be utilized in generation of an appropriate transplant. For example, cells may be derived from stem cells, such as embryonic or parthenogenic stem cells, or harvested and cultured from existing tissue. In various aspects, it is advantageous, but not necessary, for cells to be derived from the same species as the subject, if not from the subject itself. Accordingly, cells may be allogeneic, syngeneic and/or xenogeneic depending on the application.

It is appreciated that an agent as used herein, is intended to include any type of molecule capable of modulating CD44 activation, signaling or expression. For example, an agent may be an agonist or antagonist of the CD44 receptor, to either facilitate or inhibit activation or stimulation of the receptor. Alternatively, an agent may inhibit or increase expression of the CD44 receptor.

An agent or test agent is intended to include any type of molecule, for example, a polynucleotide, a peptide such as an antibody, a peptidomimetic, peptoids such as vinylogous peptoids, chemical compounds, such as organic molecules or small organic molecules, or the like, which may effect cardiomyocyte contraction. Agents encompass numerous chemical classes, though typically they are chemical compounds, such as an organic molecule, and often are small organic compounds (i.e., small molecules) having a molecular weight of more than 100 Daltons and less than about 2,500 Daltons. Agents comprise functional groups necessary for structural interaction with proteins, particularly hydrogen bonding, and typically include at least an amine, carbonyl, hydroxyl or carboxyl group, preferably at least two of the functional chemical groups. The agents may often comprise cyclical carbon or heterocyclic structures and/or aromatic or polyaromatic structures substituted with one or more of the above functional groups. Agents are also found among biomolecules including peptides, saccharides, fatty acids, steroids, purines, pyrimidines, derivatives, structural analogs or combinations thereof.

Agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In another embodiment, the present invention provides a method of screening for an agent that modulates Th1 memory cell survival. The method includes contacting or administering a test agent with a Th1 cell or population thereof, and detecting an increase or decrease in the CD44 receptor expression or signaling as compared with expression or signaling prior to contacting or administering with the agent, thereby identifying the test agent as an agent that modulates Th1 cell survival. In one aspect, the test agent is an agent that increases CD44 receptor expression or signaling, such as a CD44 receptor agonist. In another aspect, the test agent is an agent that decreases CD44 receptor expression or signaling, such as a CD44 receptor antagonist or antisense nucleic acid molecule.

The screening methods of the present invention may be performed on a number of platforms and utilize a variety of cell types. The methods of the present invention may be performed, for example using a cell based assay using the cellular composition described herein. As such, the method is particularly suited to be performed in a high-throughput fashion, (i.e., 96 or 384-well plate analysis; mechanical or robotic processing).

As discussed above, one of skill in the art would appreciate that agents that modulate CD44 receptor activity or expression include a variety of different types of molecules. An agent useful in any of the methods of the invention can be any type of molecule, for example, a polynucleotide, a peptide, a peptidomimetic, peptoids such as vinylogous peptoids, chemical compounds, such as organic molecules or small organic molecules, or the like.

Accordingly, in one aspect an agent may be a peptide or protein. The terms “polypeptide” and “protein” are used broadly to refer to macromolecules comprising linear polymers of amino acids which may act in biological systems, for example, as structural components, enzymes, chemical messengers, receptors, ligands, regulators, hormones, and the like. Such polypeptides/proteins would include functional protein complexes, such as antibodies. The term “antibody” is used broadly herein to refer to a polypeptide or a protein complex that can specifically bind an epitope of a polypeptide or antigen, such as the CD44 receptor. As used in this invention, the term “epitope” refers to an antigenic determinant on a polypeptide or an antigen, such as a cell surface marker or receptor, such as the CD44 receptor, to which the paratope of an antibody binds.

Generally, an antibody contains at least one antigen binding domain that is formed by an association of a heavy chain variable region domain and a light chain variable region domain, particularly the hypervariable regions. An antibody can be a naturally occurring antibodies, for example, bivalent antibodies, which contain two antigen binding domains formed by first heavy and light chain variable regions and second heavy and light chain variable regions (e.g., an IgG or IgA isotype) or by a first heavy chain variable region and a second heavy chain variable region (V_{HH} antibodies), or on non-naturally occurring antibodies, including, for example, single chain antibodies, chimeric antibodies, bifunctional antibodies, and humanized antibodies, as well as antigen-binding fragments of an antibody, for example, an Fab fragment, an Fd fragment, an Fv fragment, and the like.

Generally, an antibody contains at least one antigen binding domain that is formed by an association of a heavy chain variable region domain and a light chain variable region domain, particularly the hypervariable regions. Antibodies include polyclonal and monoclonal antibodies, chimeric, single chain, and humanized antibodies, as well as Fab fragments, including the products of a Fab or other immunoglobulin expression library. Antibodies which consist essentially of pooled monoclonal antibodies with different epitopic specificities, as well as distinct monoclonal antibody preparations are provided. Monoclonal antibodies are made by methods well known to those skilled in the art. The term antibody as used in this invention is meant to include intact molecules as well as fragments thereof, such as Fab and F(ab′)2, Fv and SCA fragments which are capable of binding an epitopic determinant on a protein of interest. An Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner. An (Fab′)2 fragment of an antibody can be obtained by treating a whole antibody molecule with the enzyme pepsin, without subsequent reduction. A (Fab′)2 fragment is a dimer of two Fab′ fragments, held together by two disulfide bonds. An Fv fragment is defined as a genetically engineered fragment containing the variable region of a light chain and the variable region of a heavy chain expressed as two chains. A single chain antibody (“SCA”) is a genetically engineered single chain molecule containing the variable region of a light chain and the variable region of a heavy chain, linked by a suitable, flexible polypeptide linker.

As used herein, a “monoclonal antibody” may be from any origin, such as mouse or human, including a chimeric antibody thereof. Additionally, the antibody may be humanized.

In another aspect, an agent for use in the method of the present invention is a polynucleotide, such as an antisense oligonucleotide or RNA molecule. In various aspects, the agent may be a polynucleotide, such as an antisense oligonucleotide or RNA molecule, such as microRNA, dsRNA, siRNA, stRNA, and shRNA.

The terms “small interfering RNA” and “siRNA” also are used herein to refer to short interfering RNA or silencing RNA, which are a class of short double-stranded RNA molecules that play a variety of biological roles. Most notably, siRNA is involved in the RNA interference (RNAi) pathway where the siRNA interferes with the expression of a specific gene. In addition to their role in the RNAi pathway, siRNAs also act in RNAi-related pathways (e.g., as an antiviral mechanism or in shaping the chromatin structure of a genome).

Polynucleotides of the present invention, such as antisense oligonucleotides and RNA molecules may be of any suitable length. For example, one of skill in the art would understand what length are suitable for antisense oligonucleotides or RNA molecule to be used to regulate gene expression. Such molecules are typically from about 5 to 100, 5 to 50, 5 to 45, 5 to 40, 5 to 35, 5 to 30, 5 to 25, 5 to 20, or 10 to 20 nucleotides in length. For example the molecule may be about 5, 10, 15, 16, 17, 18, 19, 20, 21, 22, 23, 24, 25, 26, 27, 28, 29, 30, 31, 32, 33, 34, 35, 40, 45 or 50 nucleotides in length. Such polynucleotides may include from at least about 15 to more than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In various embodiments antisense oligonucleotides or RNA molecules include oligonucleotides containing modifications. A variety of modification are known in the art and contemplated for use in the present invention. For example oligonucleotides containing modified backbones or non-natural internucleoside linkages are contemplated. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In various aspects modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Certain oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

In various aspects modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In various aspects, oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. In various aspects, oligonucleotides may include phosphorothioate backbones and oligonucleosides with heteroatom backbones. Modified oligonucleotides may also contain one or more substituted sugar moieties. In some embodiments oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂ and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N3, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Another modification includes 2′-methoxyethoxy(2′OCH₂CH₂OCH₃, also known as 2′-O-(2-methoxyethyl) or 2′-MOE).

In related aspects, the present invention includes use of Locked Nucleic Acids (LNAs) to generate antisense nucleic acids having enhanced affinity and specificity for the target polynucleotide. LNAs are nucleic acid in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2.

Other modifications include 2′-methoxy(2′-O—CH₃), 2′-aminopropoxy(2′-OCH₂CH₂CH₂NH₂), 2′-allyl (2′-CH—CH—CH₂), 2′-O-allyl (2′-O—CH₂—CHCH₂), 2′-fluoro (2′-F), 2′-amino, 2′-thio, 2′-Omethyl, 2′-methoxymethyl, 2′-propyl, and the like. The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrimido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases are known in the art. Certain of these nucleobases are particularly useful for increasing the binding affinity of the oligomeric compounds described herein. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2 C and are presently preferred base substitutions, even more particularly when combined with 2′-O-methoxyethyl sugar modifications.

Another modification of the antisense oligonucleotides described herein involves chemically linking to the oligonucleotide one or more moieties or conjugates which enhance the activity, cellular distribution or cellular uptake of the oligonucleotide. The antisense oligonucleotides can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., dihexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylaminocarbonyloxycholesterol moiety.

As discussed herein, contacting or administering of a subject or sample may be done in vivo by administration of the agent. The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The route of administration of a composition containing an agent as identified herein, will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known. For example, a peptide agent can be prepared using D-amino acids, or can contain one or more domains based on peptidomimetics, which are organic molecules that mimic the structure of peptide domain; or based on a peptoid such as a vinylogous peptoid. Where the agent is a small organic molecule, it can be administered in a form that releases the active agent at the desired position in the body (e.g., the stomach), or by injection into a blood vessel.

Regardless of the route of administration selected, the compounds of the present invention, which may be used in a suitable hydrated form, and/or the pharmaceutical compositions of the present invention, are formulated into pharmaceutically acceptable dosage forms such as described herein or by other conventional methods known to those of skill in the art.

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of agent to increase insulin expression or reduce lipotoxicity a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day which can be administered in single or multiple doses.

If desired, the effective daily dose of the active compound may be administered as two, three, four, five, six or more sub-doses administered separately at appropriate intervals throughout the day, optionally, in unit dosage forms. There may be a period of no administration followed by another regimen of administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Additionally, in various aspects, a physician or veterinarian having ordinary skill in the art can readily determine an appropriate subject for administration of the agents described herein. For example, one of skill in the art is capable of routine diagnosis of diabetes. Also, it is routine for one of skill in the art to determine the appropriate agents to be administered to the subject as well as the timing of administration depending on the diagnosis (e.g., Type I diabetes).

When other therapeutic agents are employed in combination with the compounds of the present invention they may be used for example in amounts as noted in the Physician Desk Reference (PDR) or as otherwise determined by one having ordinary skill in the art.

The term “effective amount” is defined as the amount of the compound or pharmaceutical composition that will elicit the biological or medical response of a tissue, system, animal or human that is being sought by the researcher, veterinarian, medical doctor or other clinician, e.g., restoration or maintenance of insulin production. For example, a “therapeutically effective amount” of, e.g., agent(s), with respect to the subject method of treatment, refers to an amount of the compound in a preparation which, when applied as part of a desired dosage regimen brings about, e.g., a change in insulin production according to clinically acceptable standards for the disorder to be treated.

The following examples are intended to illustrate but not limit the invention.

### Example 1

### Virus and Antibody Preparation

CD44^{-/-} mice are bred to B6PL-Thy mice and crossed to OT-I and OT-II TCR Tg mice. These mice are also crossed to B6 Ly5.1 mice. C57BL/6 mice are purchased from Jackson Laboratories. All mice are males between 6 and 16 weeks of age.

All influenza viruses are grown in chicken eggs (10 days of embryonation) and titrated with MDCK cells for plaque-forming units (pfu) (Jelley-Gibbs et al., J. Exp. Med., 202:697-706 (2005)). Infective doses elicit an optimal T cell response and are given i.n. in 30 ml. The WT influenza A viruses Puerto Rico/8/34 (PR8, H1N1) is given in a dose of 12.5 pfu. The engineered influenza A viruses William Smith Neurotropic/33 (WSN-OVA_{II}, H1N1) (Chapman et al., Virology, 340:296-306 (2005)) and Hong Kong Aichi/2/68 (HKx31-OVA_{II}, H3N2) (Thomas et al., Proc. Natl. Acad. Sci. USA, 103:2764-2769 (2006)) that express the OVA₃₂₃₋₃₃₉ peptide recognized by OT-II CD4⁺ T cells are given in doses of 1250 pfu and 112 pfu, respectively.

Anti-Thy1.1 (CD90.1, OX-7) and −Ly5.1 (CD45.1, A20) are from BioLegend. The following antibodies are from eBioscience: Bcl2 (10C4), Bcl-xl (2H12), and Bnip3 (polyclonal rabbit), IFN-g (XMG1.2) and TNF-a (MP6xt22). Antivb5 (MR9) and anti-Fas (CD95, Jo2) are from BD Biosciences. Anti-BimS (14A8) is from Millipore. Cell lines producing the adhesion blocking anti-CD44 mAb, KM201, and the agonist/activating anti-CD44 mAb, IRAWB 14, are obtained from P. Kincade (Zheng et al., J. Cell Bio., 130:485-495 (1995)). These mAb, anti-CD3 (145-2C11), and anti-CD28 (37.51) are produced and purified by BioLegend. Rat IgG is used as the control (Jackson ImmunoResearch).

CD4⁺ and CD8⁺ T cells are isolated from pooled spleen and lymph nodes by magnetic sorting (Imag™, BD Biosciences) according to the manufacturer's protocol. To generate effector cells, naive T cells are cultured at 10⁶/ml for 4 days with immobilized anti-CD3 (10 mg/ml) and anti-CD28 (5 mg/ml) and from TCR Tg mice with C57BL/6 splenic APCs (2-3×10⁶/ml), and 5 mM OVA peptides (257-264, OT-I and 323-339, OT-II, Sigma-Genosys) (Harbertson et al., J. Immunol., 168:1095-1102 (2002)). CD4⁺ T subsets were polarized in the presence of rIL-2 (10 ng/ml) with anti-CD3 and anti-CD28, or with APCs and peptide for 4 days. Th1 cells are elicited with rIL-12 (5 ng/ml), and anti-IL-4 (11b11, 10 mg/ml) and Th2 cells with rIL-4 (10 ng/ml) and anti-IFN-g (XMG1.2, 10 mg/ml). For Th17 cells, rIL-1b (10 ng/ml), rIL-6 (20 ng/ml), rTGF-b1 (1 ng/ml), TNF-a (10 ng/ml), and 10 mg/ml each of anti-IL-4 and anti-IFN-γ are used. Splenic DCs are enriched by centrifugation with 13.5% (w/v) histodenz (Sigma) (McLellan et al., J. Immunol. Methods, 184:81-89 (1995)).

Adoptive transfer and detection of T cells is performed as followed. T cells are injected i.v. into recipients in doses of 0.3-3.0 3×10⁶ with donor and host combinations that differed by expression of Thy1 or Ly5. WT and CD44^{-/-} cells are coinjected in equal ratios. DCs pulsed for 2 hours with 10 mg/ml OVA peptide are injected in a dose of 1×10⁵/recipient at the time of T cell transfer. After sacrifice, cells in the airways of recipient mice are collected by BAL. Cell suspensions of perfused lungs are obtained by digestion with collagenase D (Roche) at 10 mg/ml for 60 min at 37° C. Flow cytometry is used to detect Tg⁺ CD4⁺ T cells in these tissues, and in LN and spleen cells after fluorescent staining for CD4, Thy1.1, Ly5.1, and vb5 (BD Biosciences). Viable lymphocyte recovery is determined by flow cytometry with propidium iodide uptake.

T cell responses were determined as follows. CFSE or BrdU labeling (Harbertson et al., J. Immunol., 168:1095-1102 (2002); Linton et al., J. Exp. Med., 197:875-883 (2003)) are used to assess cell division. Intracellular staining is used to detect cytokine secretion by donor cells after overnight culture of lymphocyte suspensions from the MSLN with splenic APC and OVAII peptide (Harbertson et al., J. Immunol., 168:1095-1102 (2002); Linton et al., J. Exp. Med., 197:875-883 (2003)). After surface staining, the cells are permeabilized (BD Biosciences) and stained with anti-IFN-γ and TNF-α. Annexin V and 7-Amino-actinomycin D (7AAD) staining are used to distinguish dead from dying cells by flow cytometry. To test caspase 8 activity, freshly isolated MSLN cells are incubated with CaspaLux 8-L1D2 (OncoImmunin, Inc) according to the manufacturer's protocol. Fas-mediated cell death is induced with plate-bound anti-Fas (10 mg/ml). In vitro treatment of CD4⁺ T cells with anti-CD44 mAb or Rat IgG is done as soluble or plate bound (10 mg/ml) as indicated in the text.

Anti-CD44 treatment provided in vivo is performed as follows. KM201, IRAWB 14, or Rat IgG are administered in a dose of 200 mg/mouse on the day of cell transfer and three times thereafter spaced by 3 day intervals or given in a dose of 1 mg/mouse 8 days after cell transfer.

PCR for CD44 isoforms is performed as follows. OTII CD4⁺ T cells are enriched and polarized as described above and RNA isolated (RNeasy™, QIAGEN). Primers spanning the CD44 isoform region (forward Exon 5 primer: ATCAGTCACAGACCTACCCAATTCC (SEQ ID NO: 1) and reverse exon 16 primer CCAAGATGATGAGCCATTCTGGAATC (SEQ ID NO: 2)) are used to distinguish isoforms by RT-PCR (GeneAmp™, Applied Biosystems). Reverse transcriptase is for 60 minutes at 42° C. via random hexamers and reverse primer, followed by PCR with the following conditions: 94° C. 2 minutes, 64° C. 30 seconds, 72° C. 1 minute 30 seconds (35 cycles).

Immunoblots for Akt are performed as follows. CD4+ cells from WT or CD44^{-/-} mice are stimulated with plate-bound anti-CD3 plus anti-CD28 (5 mg/ml each) under Th1 or Th2 cell conditions for 4 days, followed by 2 days of resting in RPMI culture medium with or without rIL-7 for the first 24 hours. To crosslink CD44, 10⁶ cells are plated on IRAWB 14- or rat IgG-coated plates, incubated at 37° C. for 15, 30, 60, or 120 minutes. After stimulation, cell lysate proteins are resolved through 4%-12% PAGE and transferred to nitrocellulose membranes. The membranes are probed and reprobed with Abs to phospho-Akt and p85 subunit of PI3K (Cell Signaling Technology), respectively. Densitometry on blots was performed with ImageJ™ (NIH).

### Example 2

### Loss of CD4⁺ T Cell Memory in the Absence of CD44

To investigate the role of CD44 in the development of immunity, an influenza model is utilized in which viral clearance from the lung epithelium depends upon a local T cell response. In an initial comparison of wild-type (WT) and CD44-deficient (CD44^{-/-}) mice, it is determined that CD4⁺ and CD8⁺ lymphocyte subsets are normally represented in CD44^{-/-} mice, because of additional compensatory HA binding receptors. Moreover, differences in expression of several adhesion receptors, including CD62L, the integrins CD11a and CD49, CD45RB, and CD69 on CD4⁺ T cells from 6-month-old animals are not observed. Since CD44 is expressed by multiple cell types, the role of CD44 in CD4⁺ T cells is directly assessed with WT and CD44^{-/-} mice crossed to OT-II TCR transgenic (Tg) mice whose CD4⁺ T cells recognize a peptide of ovalbumin (OVA_{II} or OVA₃₂₃₋₃₃₉). Naive WT and CD44^{-/-} Tg CD4+ T cells marked by expression of the Vb5 chain of the TCR and by the allelic variants of Ly5.1 (CD45.1) or Thy1.1 (CD90.1), respectively, are isolated by negative selection from the lymphoid tissues of 6-week-old donors.

The WT and CD44^{-/-} cells are coinjected in equal numbers into normal C57BL/6 mice (Thy1.2, Ly5.2) to provide an internal control for the response. The cells are titrated to the lowest number needed for a consistently detectable response (3×10⁵), where all cells became engaged as measured by division that was detected by CFSE dilution. The recipients are then infected with the William Smith Neurotropic (WSN) influenza A virus (H1N1) expressing the OVAII peptide (WSN-OVA_{II}) (Chapman et al., Virology, 340:296-306 (2005)). CD4⁺ T cell memory development is assessed by challenging recipients 3 weeks later with the recombinant influenza virus Hong Kong Aichi. 2.68×31 (H3N2) that also expressed the OVAII peptide (HKx31-OVA_{II}) (Thomas et al., Proc. Natl. Acad. Sci. USA, 103:2764-2769 (2006)).

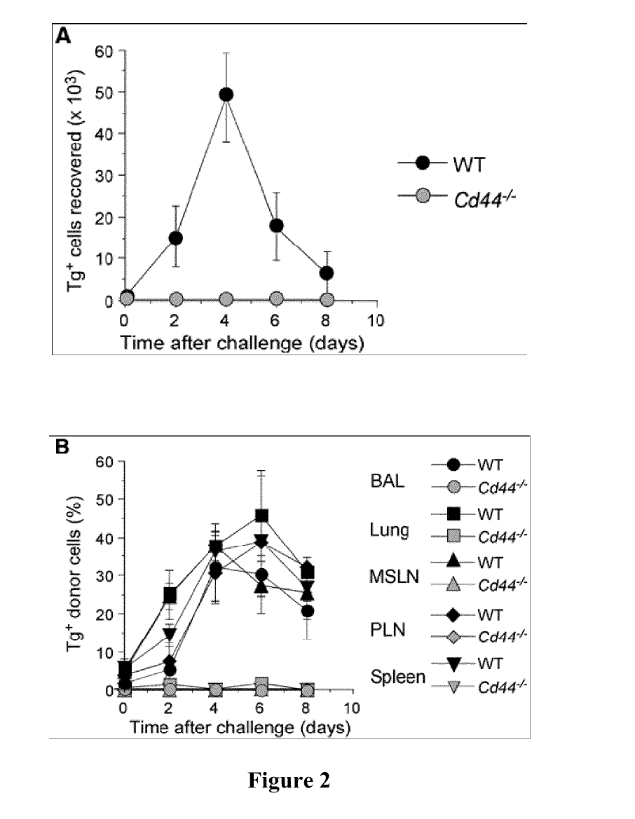

The kinetics of cell expansion is evaluated as measured by the recovery of donor cells. At the time of challenge, the frequencies of donor cells are very low. A memory response is observed from WT OT-II donor cells in the draining mediastinal lymph nodes (MSLN) as indicated by a >50-fold increase in the number of Tg⁺ CD4⁺ T cells on day 4, the peak of the response (FIG. 2A).

FIG. 2 shows requirement for CD44 in the generation of memory responses in CD4⁺ T cells. CFSE-labeled OT-II cells from WT (Ly5.1) and CD44^{-/-} (Thy1.1) mice are cotransferred (3×10⁵ each) into C57BL/6 recipients (Ly5.2, Thy1.2) that are then infected with WSN-OVA_{II}. After 22 days, the recipients are challenged with HKx31-OVAII. FIG. 2A shows the recovery of Tg+ WT and CD44^{-/-} cells in the MSLN from individual animals. FIG. 2B shows the percentage Tg⁺ cells in the Vb5⁺, CD4+ population from BAL, lung, MSLN, PLN, and spleen (mean±SEM, n=3-4/group).

Dramatic increases in the frequencies of WT OT-II cells are also observed in the airways (retrieved by bronchoalveolar lavage, BAL) and lungs, as well as other lymphoid tissues (peripheral lymph nodes, PLN, and spleen) (FIG. 2B). However, CD4⁺ T cells from the CD44^{-/-} OT-II donors are undetectable in the MSLN (FIG. 2A) and are present only in very low numbers systemically (FIG. 2B). The present invention provides that expressions of CD44 on CD4⁺ T cells are necessary for either the appropriate development of memory cells or their capacity for expansion.

### Example 3

### Unimpaired Induction of CD4⁺ T Cell Responses in the Absence of CD44

CD44 is know to regulate T cell migration via interactions with vascular endothelium through HA, which acts to initiate extravasation into tissue. Therefore, the absence of CD44 could generally affect the trafficking of CD44^{-/-} CD4⁺ T cells. Thus, the recovery of nai\ddot{v}e CD44^{-/-} and WT CD4⁺ T cells with time after transfer to unimmunized hosts is evaluated.

The presence of comparable numbers of naive and WT cells in the lymphoid compartment and no differences in their distribution suggest normal homeostatic regulation and migration. Since inflammation could affect CD4⁺ T cell trafficking and since cells from CD44^{-/-} mice display the normal responses to TCR activation in vitro, whether the failure of CD44^{-/-} CD4⁺ T cells to generate a memory effector population can be attributed to defects in trafficking of naive or effector cells after influenza virus infection is evaluated. Homing of naive or in vitro activated OT-II effector CD4⁺ T cells to either lymphoid or nonlymphoid tissues is unimpaired by the absence of CD44 irrespective of whether the recipients are naive or are infected several days previously. Thus, although CD44 can regulate homing of CD4⁺ T cells, this function is not essential and/or is replaceable by other adhesion receptors.

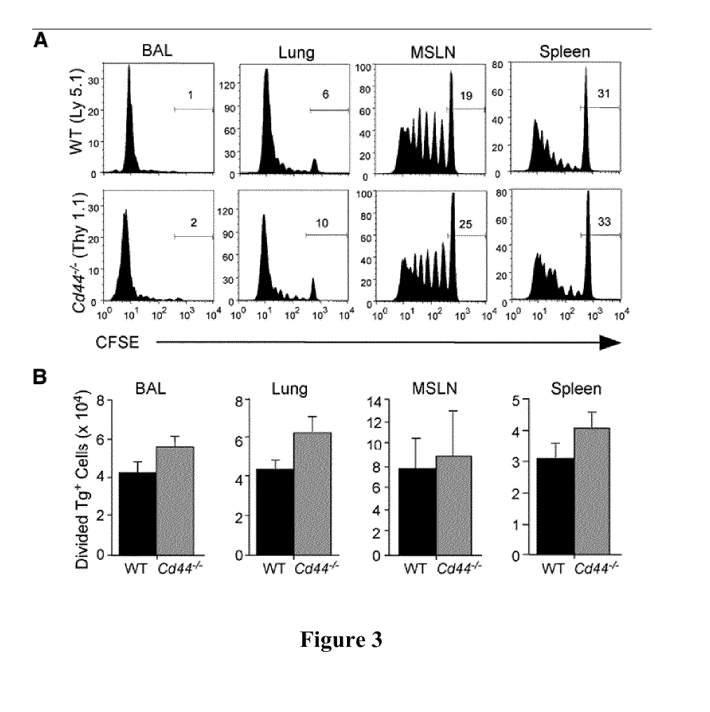

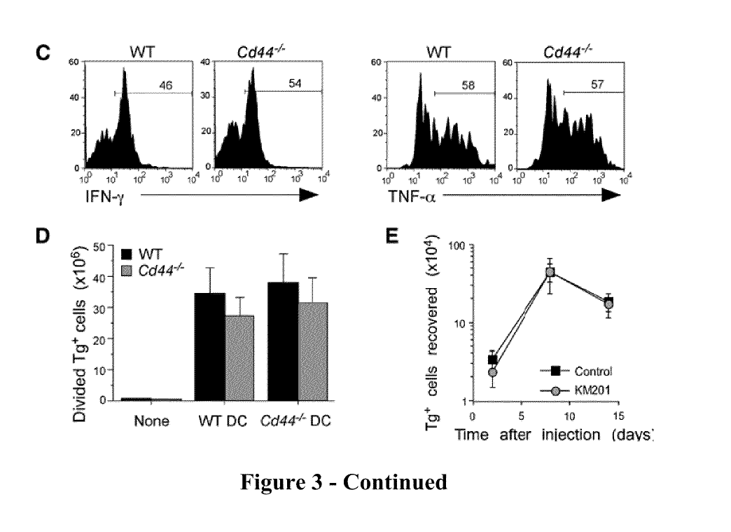

Although differences in the responses of CD44^{-/-} and WT OT-II cells to TCR stimulation with peptide and antigen-presenting cells (APCs) are not observed in vitro, the present invention provides that CD44 contributes to the initial priming or responses of CD4+ T cells in vivo. Therefore, the division of adoptively transferred WT and CD44^{-/-} Tg⁺ cells is analyzed by CFSE dilution at the peak of the response to WSN-OVA_{II}. Here, A higher dose of Tg+ cells to observe potential differences in the kinetics of division is utilized. Similar kinetics of division in WT and CD44^{-/-} cells (FIG. 3A) and distribution of cells in the lymphoid tissues and lungs as measured by recovery (FIG. 3B) are observed. FIG. 3 shows CD44 independence of CD4+ T cell priming. C57BL/6 mice are injected with CFSE-labeled WT and CD44^{-/-} OT-II cells (1.5×10⁶ each) and infected with WSN-OVA_{II}. After 8 days, division of Tg⁺ cells is analyzed by CFSE (FIG. 3A). The marker on each histogram shows the fraction of undivided cells. FIG. 3B shows the average recoveries of Tg⁺ donor cells that underwent one or more divisions on day 8 (mean±SEM, n=3-4/group). This suggests that CD44 is not essential for either the induction of a CD4+ T cell response to influenza virus or for the migration of CD4⁺ T cells. Furthermore, both populations exhibit a similar capacity to develop effector function as measured by secretion of IFN-γ and TNF-α (FIG. 3C). FIG. 3C shows IFN-γ and TNF-α production by WT and CD44^{-/-} cells after overnight restimulation by OVA_{II} peptide with splenic APC.

Additionally, it has been confirmed that expression of CD44 is not required on either CD4+ T cells or DCs to initiate a primary response in vivo, as shown by the induction of comparable numbers of dividing WT and CD44^{-/-} cells after immunization with OVA peptide-pulsed WT or CD44^{-/-} splenic DCs (FIG. 3D). CFSE-labeled WT and CD44^{-/-} OT-II cells are coinjected into C57BL/6 recipients as for (A) together with 2 3 105 CD11c⁺, OVAII peptide-pulsed DCs from either WT or CD44^{-/-} C57BL/6 mice. FIG. 3D shows recovery of donor CD4+ T cells that had undergone one or more divisions in the spleen 4 days later (mean±SEM, n=4/group).

To further investigate whether blocking CD44 during Th1 cell priming can predispose the cells to death, OT-II cells are treated with the CD44 adhesion-blocking mAb, KM201, during in vitro culture with APCs and peptide under Th1 cell polarizing conditions. Cell recoveries at day 4 were similar to the isotype control-treated cultures, and exposure to KM201 do not negatively affect the ability of Th1 cells to persist (FIG. 3E). WT OT-II Th1 cells are generated with APC and OVA_{II} peptide in the presence of the blocking anti-CD44 mAb, KM201, or control IgG. The cells are then injected into separate groups of C57BL/6 recipients (2×10⁶/mouse). The donor Tg⁺ cells recovered in the pooled lymph node (LN) and spleens of mice are shown in FIG. 3E at the indicated times after injection (mean±SEM, n=3-4/group). The results show that initial activation, expansion, and effector development proceed normally in CD44^{-/-} CD4⁺ T cells.

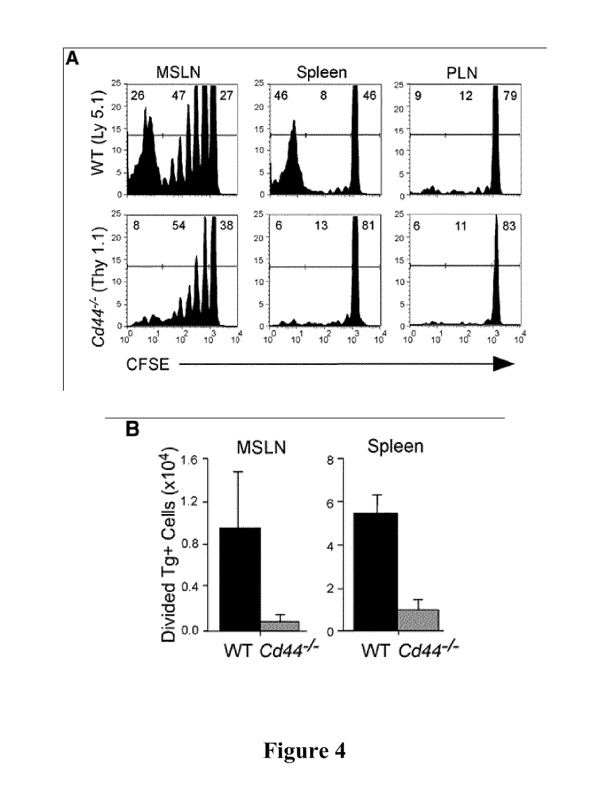

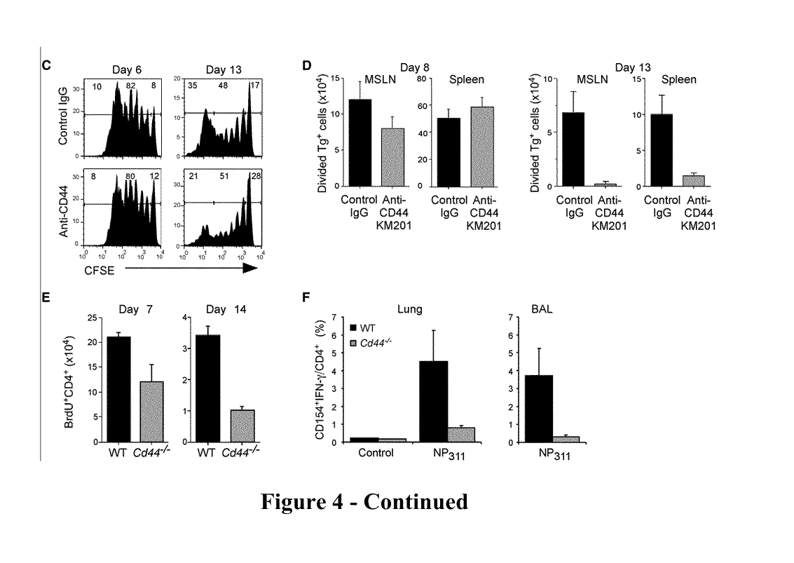

These findings suggest that the mechanisms which limit the development of memory in CD4⁺ T cells could become manifest at the later stages of the primary response. Therefore, cell division is analyzed at 13 days after infection. Divided CD44^{-/-} CD4⁺ T cells fail to accumulate in the lymphoid tissues and are not detected in the lungs (FIGS. 4A and 4B). Loss of CD4⁺ T cell effectors in the absence of CD44 engagement is evidenced in FIG. 4. C57BL/6 recipients were injected with CFSE-labeled WT and CD44^{-/-} OT-II cells (1.5 3 106 each) and infected with WSN-OVA_{II}. For FIGS. 4A and 4B, on day 13 after infection, division (A) and recovery (B) of Tg⁺ cells is determined as for FIG. 3 in the MSLN and spleen.

In order to prevent ligand binding by CD44, the KM201 mAb is administered, beginning at the time of WT cell transfer and infection. This does not affect the recovery of divided cells on day 6, but the accumulation of responding CD4⁺ T cells on day 13 is inhibited when compared to mice that received control IgG (FIGS. 4C and 4D). For FIGS. 4C and 4D, recipients of CFSE-labeled WT OT-II cells are injected with either KM201 anti-CD44 or control IgG at the time of cell transfer and infection with WSN-OVA_{II} influenza virus and three more times at 3 day intervals. FIG. 4C shows the division of the donor cells on days 6 and 13, while FIG. 4D shows recovery of donor CD4⁺ T cells that have undergone one or more divisions in the MSLN and spleen 8 and 13 days after infection.

This outcome is unlikely to be due to cytotoxic effects, because KM201 and several other anti-CD44 reagents do not elicit complement-mediated killing of activated OT-II cells. The results suggest that CD44 participates in mechanisms that promote the survival of activated CD4⁺ T cells. Since the model used involves transfer of a relatively large number of Tg⁺ CD4⁺ T cells that might affect regulation, the responses of endogenous CD4⁺ T cells from the lungs and MSLN of WT and CD44^{-/-} mice are assessed (Schmits et al., Blood, 90:2217-2233 (1997)) after infection with influenza virus with BrdU uptake (FIG. 4E). The results show that fewer divided CD4⁺ T cells are present in the MSLN of CD44^{-/-} mice compared to WT mice on day 14. In addition, the lungs and BAL contain fewer virus-specific CD44^{-/-} CD4⁺ T cells than WT CD4⁺ T cells on day 21 (FIG. 4F).

For FIGS. 4E and 4F, WT and CD44^{-/-} mice ice are infected with PR8 influenza virus. The mice are treated with BrdU for 7 days before sampling. The recovery of BrdU⁺ CD4⁺ T cells is shown in FIG. 4E. On day 21 after infection, the virus-specific CD4⁺ T cell response is assessed by intracellular staining of cells from the lungs and BALs after overnight culture with anti CD28 in the presence or absence of NP311 peptide. Shown in FIG. 4F are the percentages of CD154⁺IFN-γ⁺ virus-specific CD4⁺ T cells in the lung. For FIGS. 4B, 4D and 4F, mean±SEM, n=3-4/group.

### Example 4

### Responding CD44^{-/-} CD4⁺ Cells Die by Apoptosis

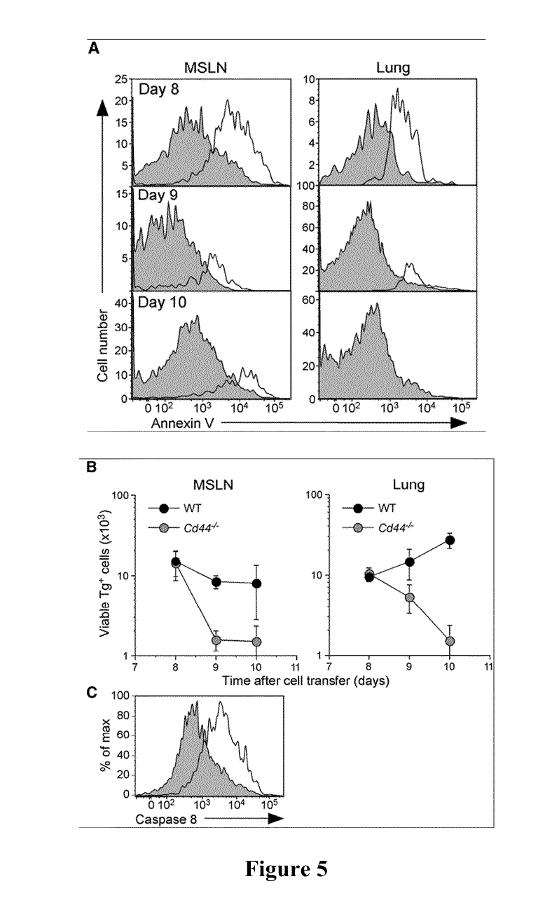

To assess whether apoptosis is evident in CD44^{-/-} CD4 T cells during expansion after infection, Annexin V binding is examined in cells from the MSLN and lungs (FIG. 5A). Apoptotic cells are observed on day 8, followed by the disappearance of responding cells on day 9 (FIG. 5B). C57BL/6 recipients are given WT and CD44^{-/-} OT-II cells (1.5×10⁶ each) and infected with WSN-OVA_{II}. For FIG. 5A, apoptosis is assessed by binding of Annexin V and exclusion of 7AAD by WT Tg⁺ cells (shaded histograms) and CD44^{-/-} Tg⁺ cells (open histograms) in the indicated tissues. FIG. 5B shows viable recoveries of WT and CD44^{-/-} donor Tg⁺ cells in the MSLN and lungs (mean±SEM, n=5/group).

Since CD44 has been associated with resistance to Fas-mediated cell death in various cell lines and tumor cells, the activation of intracellular caspase 8 in CD44^{-/-} CD4⁺ T cells is quantified. Activation is known to characterize the induction of extrinsic apoptotic death and occurs as a consequence of the assembly of the death-inducing signaling complex (DISC). On day 7 after adoptive transfer, activated caspase 8 is markedly increased in CD44^{-/-} cells compared to WT cells from the MSLN (FIG. 5C). For FIG. 5C, caspase 8 activation is assessed with a fluorophore-modified substrate in dispersed MSLN cells from recipients of WT and CD44^{-/-} CD4⁺ T cells on day 7 after infection. The fluorescence induced by activated caspase 8 for WT Tg⁺ cells (shaded histogram) and CD44^{-/-} Tg⁺ cells (open histogram) is shown in 7AAD⁻ Tg⁺ population. The results are representative of those from six recipients. Although changes in Bcl-2 family members are known to correlate with the loss of CD44 and cell death in various cell types in vitro, no altered expression of the proapoptotic members Bim and BNIP-3 or the antiapoptotic members Bcl-2 and Bcl-xl is detected. The present invention provides that death receptors participate in the mechanism of CD44^{-/-} CD4⁺ T cell death in vivo and a previously unidentified role of CD44 in maintaining CD4⁺ effector T cells engaged in an immune response.

### Example 5

### CD44 can Regulate CD4⁺ T Cell Survival in the Absence of Antigen

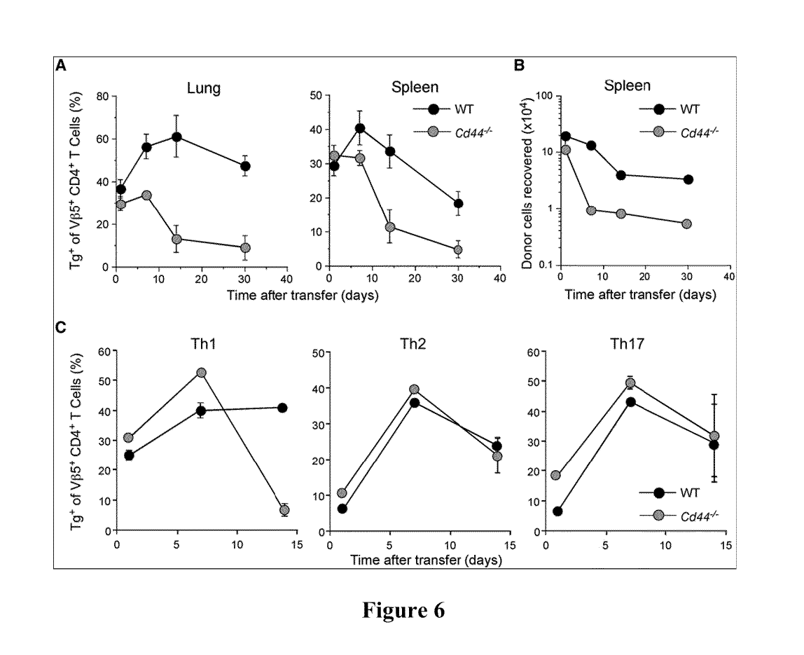

After influenza virus infection, antigen presentation to CD4⁺ T cells is known to be prolonged, raising the possibility that TCR-mediated activation-induced cell death (AICD) occurs in the absence of CD44. The present invention provides that death of CD44^{-/-} CD4⁺ T cells can occur in the absence of overt signaling by antigen. To this end, activated effectors are generated from WT and CD44^{-/-} OT-II cells in culture. Both populations undergo comparable expansion and are similarly activated as measured by size. Although WT and CD44^{-/-} effector cells show similar distribution 1 day after coinjection into normal recipients, CD44^{-/-} CD4⁺ T cells decay more quickly than WT cells in the lungs and spleen (FIG. 6A), as well as in the PLN, liver, and bone marrow. For FIG. 6A, WT and CD44^{-/-} OT-II cells are stimulated in vitro with APC and OVA peptide and then coinjected (1.5×10⁶ each) into naive C57BL6 recipients. The frequencies of Tg⁺ cells, gated on the Vb5⁺, CD4⁺ population in the lungs and spleen at the indicated times after cell transfer, are shown (mean±SEM, n=4/group).

To confirm that impaired survival is not unique to Tg⁺ cells or the viral response, it is necessary to examine the decay of CD44^{-/-} effector CD4⁺ T cells that are generated from polyclonal naive cells in vitro. Decay of CD44^{-/-} CD4⁺ T cells compared to WT cells is pronounced (FIG. 6B). The present invention provides that activated CD4+ T cells that are unable to engage CD44 ligands fail to survive in the absence of CD44. For FIG. 6B, polyclonal, non-Tg WT CD4+ T cells (Thy1.1, Ly5.2) and CD44^{-/-} CD4⁺ T cells (Thy1.2, Ly5.2) are stimulated with anti-CD3/anti-CD28 and coinjected into Ly5.1, Thy1.2 recipients in a dose of 1.5×10⁶/recipient. The recovery of donor cells in the spleen is shown (mean±SEM, n=4/group).

Since CD4⁺ T cells are known to develop into distinct subsets specified by different transcription factors and signature cytokines, and the response to influenza virus is Th1 cell biased, the present invention provides that the polarization of CD4⁺ effector T cells can affect CD44-dependent regulation of apoptosis. Th1, Th2, and Th17 WT and CD44^{-/-} cells are generated from naive CD4⁺ T cells with the appropriate cytokines The effector phenotypes are confirmed by cytokine analysis with production of IFN-γ and TNF-α by Th1 cells, IL-17 by Th17 cells, and IL-4 and IL-10 by Th2 cells. The CD4⁺ T cell subpopulations are similarly activated as indicated by the expression of CD11a and CD69. WT and CD44^{-/-} effectors of each subset are coinjected into C57BL/6 recipients. The donor cell recoveries at days 1, 7, and 14 reveals that only Th1 cells are affected by the absence of CD44 (FIG. 6C). For FIG. 6C, Th1, Th2, and Th17 cells are generated from OT-II cells with APC and OVA_{II} peptide. Allelically marked WT and CD44^{-/-} cells of each of the corresponding subsets are coinjected in a dose of 1.5×10⁶ each into C57BL/6 recipients. Shown are the frequencies of donor cells recovered at the indicated times after cell transfer after gating on the Vb5⁺, CD4⁺ population.

With Th1 and Th2 WT and CD44^{-/-} cells, no differences are observed in their ability to home to lymphoid tissues or to localize in the T cell areas of the spleens. The present invention provides that CD44 plays a role in the survival of CD8⁺ T cells. TCR Tg⁺ OVA-specific OT-I or polyclonal CD8 effectors are generated from WT and CD44^{-/-} mice. In contrast to Th1 cells, the decay of CD8⁺ T cells in unimmunized recipients is not altered by the absence of CD44. Thus, the present invention provides that CD44 regulates CD4⁺ Th1 cells distinctly and plays a nonredundant role in the regulation of the Th1 cell response and ultimately their development into memory cells.

### Example 6

### CD44 Limits Fas-Induced Cell Death in the Th1 Cells

The differential requirements of the CD4+ T cell subsets can not be attributed to CD44 expression, because this is equal between Th1 and Th2 cells. Th1 and Th2 cells are generated from WT and CD44^{-/-} OT-II Thy1.1 cells with OVA_{II} peptide and APC and tested for expression of CD44.

It has been established that Th1 cells differ from Th2 cells with respect to regulation of apoptosis and differential expression of CD44 isoforms has previously been associated with protection against death of various cell lines, including T cell lines. However, no splice variants are observed in Th1 and Th2 cells; only the standard form of CD44 is detected. Th1, Th2, and Th17 cells are generated from WT and CD44^{-/-} OT-II cells. RNA is isolated and tested for the presence of CD44 splice variants by RT-PCR with primers for the constant regions that flank the variant region. CD44 standard is 428 bp in size. In addition, no major differences in glycosylation that can lead to differences in the sizes of CD44 on Th1 versus Th2 cells are observed.

CD44 has been associated with regulation of Fas (CD95)-mediated cell death. Therefore expression of Fas and CD44 on Th1 and Th2 cells is compared. Th1 cells expressed much higher amounts of Fas than Th2 cells, a difference that is also evident with CD44^{-/-} Th1 and Th2 cells.

Furthermore, incubation of in vitro-polarized CD4⁺ T cells with anti-Fas elicit apoptosis in CD44^{-/-} Th1 cells, but not Th2 or Th17 cells. Th1, Th2, and Th17 cells are generated from WT and CD44^{-/-} CD4⁺ T cells and recultured overnight in the presence or absence of plate-bound Fas mAb. The numbers after the colored bars indicate the percentages of cells undergoing apoptosis as indicated by binding of Annexin V and exclusion of 7AAD. This difference in Th1 cell susceptibility to apoptosis is detectable by 3 hours after Fas ligation. Thus, the present invention provides that CD44 and Fas can interact to limit death of Th1 cells by affecting extrinsic death receptor engagement or signaling. However, no physical association of these molecules has been detected in the membranes of Th1, Th2, or Th17 cells by immunoprecipitation or fluorescence microscopy.

### Example 7

### CD44 Regulates Th1 Cell Survival by Engaging the PI3K-Akt Signaling Pathway

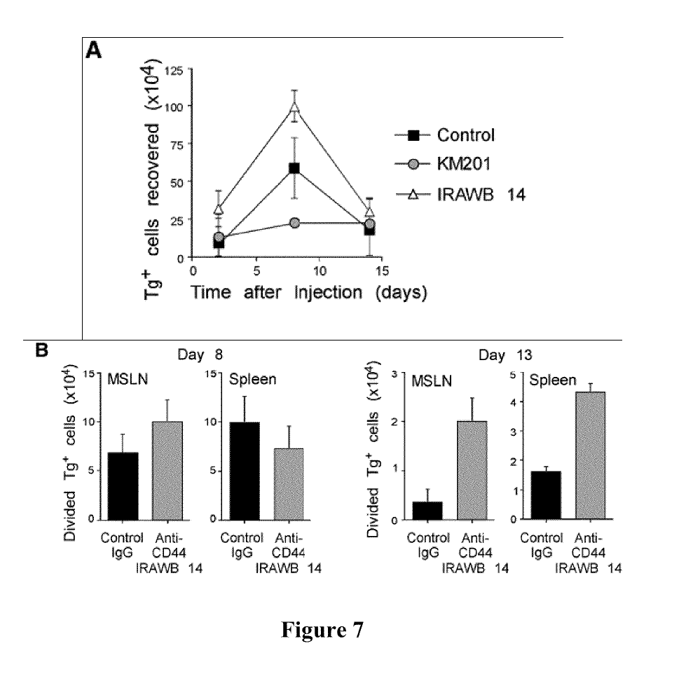

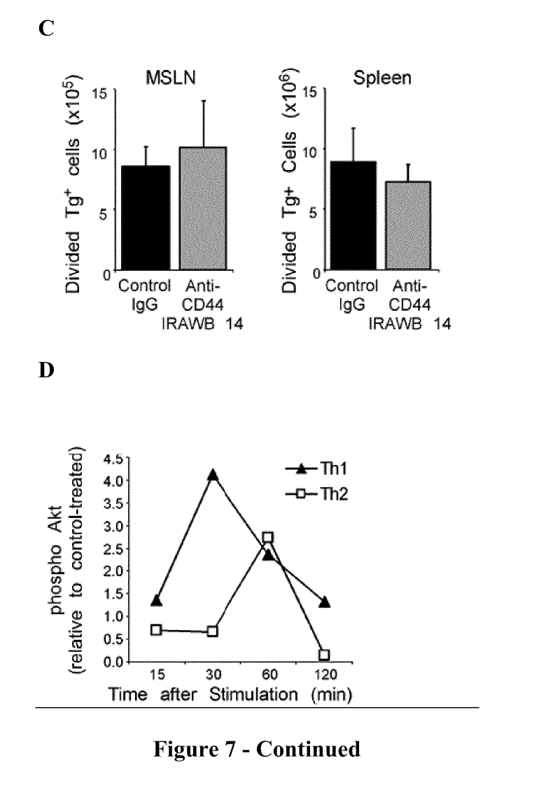

The present invention provides that signals contribute to CD4⁺ T cell survival can be initiated by CD44. Therefore, the recoveries of Th1 effector cells after transfer to unimmunized recipients that are treated with the agonist CD44 agonist antibody, IRAWB 14 or with the CD44 blocking antibody KM201 is examined. Compared to the IgG control, greater or reduced persistence of Th1 cells is observed after treatment with IRAWB 14 or KM201, respectively (FIG. 7A). For FIG. 7A, WT OT-II Th1 cells are generated with APC and OVAII peptide. The cells are then injected into C57BL/6 recipients (1.5×10⁶/mouse) and treated with control IgG, KM201, or IRAWB 14 on the day of cell transfer, and three more times at 3 day intervals. The donor Tg+ cells recovered in the pooled LN and spleens are shown at the indicated times after injection. Thus, for Th1 cells, blocking CD44 binding to its ligand(s) inhibits survival, whereas signaling through CD44 enhances survival, implying a direct effect of CD44 engagement.

Mice are treated with IRAWB 14 or control IgG at the time of transfer of WT OT-II cells and infection with the influenza virus. After IRAWB 14 treatment, there is a greater recovery of CD4⁺ T cells engaged in the response by division as compared to the controls on day 13 after infection (FIG. 7B). For FIG. 7B, C57BL/6 recipients of 1.5 3 106 CFSE-labeled WT OT-II cells are injected with either IRAWB 14 or control IgG at the time of cell transfer and infection with WSN-OVA_{II} influenza virus. The antibodies are administered three more times at 3 day intervals. The recoveries of donor CD4⁺ T cells that have undergone one or more divisions in the MSLN and spleen are measured 8 and 13 days later.

The present invention provides that ligation of CD44 can promote the accumulation of CD4⁺ T cells either by effects on survival, expansion, or both. However, IRAWB 14 does not promote proliferation of in vitro activated WT OT-II cells, and other studies support the concept that ligation of CD44 without TCR signaling does not promote division of T cells. Thus, the data suggest that CD44 participates in maintaining survival of effector cells engaged in the response to influenza virus. To determine whether CD44 is required during the expansion versus contraction phase of the CD4⁺ T cell response to influenza virus, IRAWB 14 or control Ig treatment is initiated on day 8 after WT OT-II cell transfer and infection of the recipient animals. No differences in the recoveries of OT-II cells are observed under these conditions on day 10 of the response (FIG. 7C). For FIG. 7C, C57BL/6 recipients of 1.53106 CFSE-labeled WT OT-II cells are injected with either IRAWB 14 or control IgG 8 days after infection with WSN-OVA_{II} influenza virus. The recoveries of donor CD4⁺ T cells that have undergone one or more divisions in the MSLN and spleen are measured at 10 days after infection. The present invention provides that CD44-regulated survival signals are engaged during CD4⁺ T cell expansion.

Ligation of CD44 is known to lead to activation of the PI3K-Akt pathway in some cell types and can inhibit Fas-mediated CD4⁺ T cell death by interfering with DISC assembly. To determine whether ligation of CD44 might differentially signal in T cell subsets, Th1 and Th2 cells are generated from WT OT-II cells. After resting for 2 days, the cells are cultured with plate-bound IRAWB 14 or with the control IgG antibody. Phosphorylated Akt is measured as a downstream readout of PI3K activation. Phosphorylated Akt was induced in Th1 cells with peak expression of 30 minutes after ligation of CD44, whereas this response is lower in Th2 cells and was not observed until 60 minutes. Th1 and Th2 cells are generated from WT and CD44^{-/-} C57BL/6 CD4⁺ T cells by stimulation with plate-bound anti-CD3 and anti-CD28. After resting for 1 day in rIL-7 and a further day without, the cells are cultured for 15, 30, 60 and 120 minutes with plate-bound IRAWB 14 mAb. Phospho-Akt is detected by immunoblot and compared to the p85 subunit of PI3K as a loading control. Densitometry of phospho Akt on immunoblot data and shown in FIG. 7D. Results are represented as a ratio between band densities for IRAWB 14 and unstimulated control cells and are corrected for loading differences.

To confirm the CD44 dependence of PI3K induction, Akt phosphorylation in WT Th1 cells is compared to CD44^{-/-} Th1 cells in response to IRAWB 14. PI3K is engaged only in the WT Th1 cells and expression could be reduced with the PI3K inhibitor Ly294002. The present invention provides that CD44 engagement elicits signaling that promotes survival in Th1 cells, which may be crucial in this subset because of the high expression of Fas and the associated greater susceptibility to apoptosis by this pathway.

### Example 8

### Function of CD44 in the Regulation of Memory Generation in Th1 CD4⁺ T Cells

In this example, a critical function of CD44 in the regulation of memory generation in Th1 CD4⁺ T cells is identified. Despite potential roles in migration and interactions with DCs, CD44 is not required for the initial induction of a primary immune responses in vivo or for the localization of naive or effector cells in either lymphoid or nonlymphoid tissues. This is likely because of redundancies in adhesion receptor usage that enable T cells to bypass its contribution and/or the ability of other HA binding receptors to perform these functions. However, CD44 plays a nonredundant role in regulating the survival of CD4⁺ effector T cells in the influenza model, which is dominated by a Th1 cell response. Without engagement of CD44, effector cells that have progressed through several rounds of division died by apoptosis, whereas agonist signaling via CD44 during the expansion phase led to enhanced in vivo accumulation of effector cells. Thus, the generation of a memory population in Th1 cells most probably depends upon engagement of CD44 on responding effectors during the primary response. It has been shown that CD44 ligation activates the PI3K-Akt signaling pathway in Th1 cells. The present invention provides that the mechanism by which CD44 activates PI3K can be due to constitutive association with the Src family kinases Lck and Fyn or to associations with b1 integrins that mediate the survival response.

Th1 cells may uniquely require this survival signal through CD44 because of elevated Fas expression and an inherent ability to rapidly assemble the DISC in response to Fas trimerization. Thus, it is suggested that without engagement of CD44, the response to Fas ligation cannot be overcome. Such a mechanism may not be necessary in Th2 cells, and possibly other subsets of T cells, because of overall lower expression of Fas in addition to a greater capacity to engage PI3K-Akt in response to TCR signaling or costimulation. It is of significance that activation of PI3K-Akt is known to block DISC formation by preventing the association of FADD and recruitment of pro-caspase 8 in CD4⁺ T cells, and the present example favor this mechanism for regulation of Th1 cell survival.

Although CD44 is known to mediate resistance of tumor cells to apoptosis by death receptor ligation via FasL-Fas, DR5-TRAIL, and TNFR1-TNF-α by interfering with DISC assembly through the physical association of Fas and CD44, this interaction occurs through variant isoforms. Isoforms that include variants v6 and v9 are in close proximity with Fas in the membranes of transfected Jurkat cells and thereby prevent Fas trimerization. However, the lack of CD44 isoforms on CD4+ T cells activated in vivo after influenza virus infection or on Th1, Th2, or Th17 cells generated in vitro, which differ in their susceptibility to Fas-mediated death in the absence of CD44, further argues against sequestration of Fas as the only mechanism that accounts for a selective function of CD44 in Th1 cells. The lack of CD44 isoforms on CD4⁺ T cell subsets also argues against a mechanism whereby osteopontin binding to CD44 variants containing v7 leads to activation of NF-kB and prevents mitochondrial death controlled by the transcription factor Foxo3a, a regulator of Bim. Indeed, no changes of either pro- or antiapoptotic Bcl-2 family proteins in WT compared to CD44−/− CD4⁺ T cells is detected. Although differences in glycosylation of Th2 cells have been reported to account for resistance to cell death compared to Th1 and Th17 cells, the mechanism involves protection from binding of galectin-1, and no differences in the molecular weight of CD44 from Th1 and Th2 cells are observed which suggest significant differences in glycosylation. Since CD44 is upregulated on activated and memory CD8 cells, it is unlikely that differences in their regulation that would suggest independence from CD44-mediated survival signals. However, there are many differences reported in the regulation of CD4⁺ and CD8⁺ T cells, including in the programming to develop into memory cells after the initiation of a response. The present invention provides that internal signaling differences rather than external molecular variations account for the differences in regulation by CD44 on T cells.

A role for CD44 in regulating survival of CD4⁺ T cells engaged in an immune response in vivo has not been previously examined directly. However, protection from TCR-mediated AICD by CD44 has been suggested by in vitro studies of in vivo primed cells. The results described herein, which show normal priming of CD4⁺ T cells in vivo irrespective of the presence of CD44 on T cells or DCs, support the concept that engagement of CD44 in vivo is required for Th1 cells only after activation. The results of the present example suggest that survival signals are transmitted in Th1 cells during the expansion phase of the effector response to influenza virus, which is profoundly compromised in the absence of CD44 or when adhesion binding of CD44 is blocked.

A CD44-dependent survival mechanism remained operative in activated Th1 cells that are withdrawn from overt Ag stimulation by transfer into naive recipients. This result suggests that TCR signaling in the context of an effector response and the production of proinflammatory cytokines, both of which are known to augment CD44 binding of HA, are not necessary for the function of CD44 in promoting apoptosis resistance. Indeed, in the absence of an immune response, agonist engagement of CD44 in vivo can promote enhanced accumulation of CD4⁺ T cells. Previous studies indicate that maintaining effector survival through costimulation can be key to the generation of robust memory in CD4⁺ T cells. In this regard, we propose that CD44 can be viewed as an ECM-dependent, Th1 cell-specific “costimulatory” molecule that sustains effector cell responses through survival and thereby supports the development of memory.

The homeostasis of many cell types is regulated by contact dependence, and signals from the ECM can be crucial to prevent cells from undergoing anoikis or programmed cell death, which can be due to intrinsic death resulting in mitochondrial permeabilization, and extrinsic death that is initiated by death receptors. Although the molecular mechanisms that lead to PI3K activation and the downstream targets in Th1 cells remain to be defined, the results of the current example support the concept that physical contacts of Th1 cells with HA in the immediate environment regulate processes during which CD44-dependent survival signals are engaged. By promoting optimal survival of effector CD4⁺ T cells engaged in an immune response (clonal burst), CD44 provides a previously unknown contribution to the development of T cell immunity in vivo.

### Example 9

### Targeting CD44 Leads to Selective Apoptosis of Th1 Cells in Type 1 Diabetes

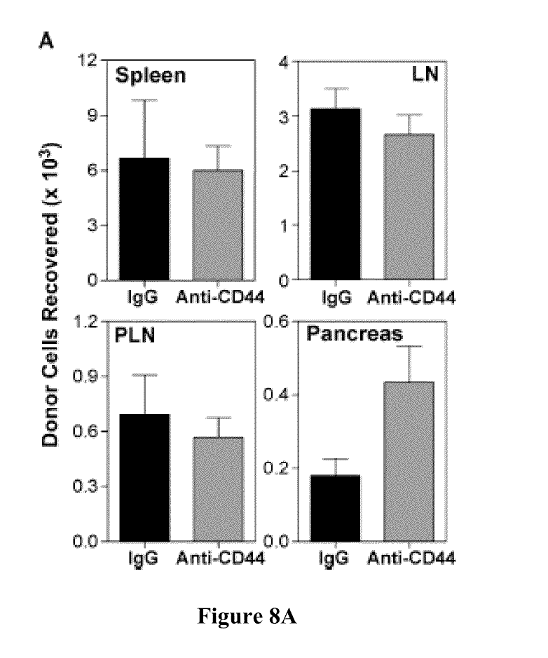

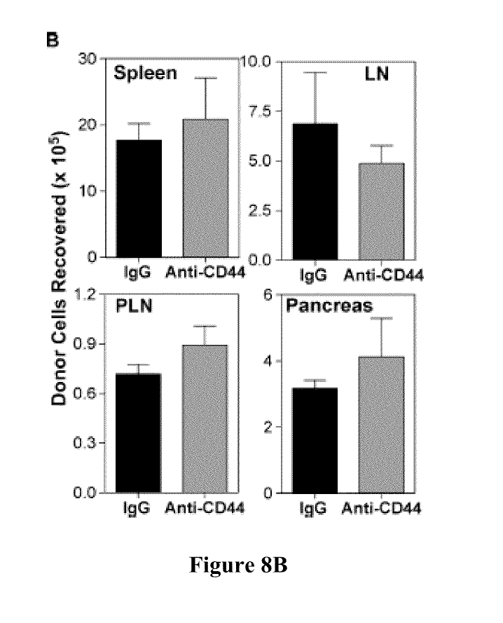

The present invention provides that CD44−/− CD4 T cells fail to generate memory in a mouse model of influenza infection. The mechanism to be selectively intrinsic to Th1 cells and independent of homing to the lungs has been identified and provided. Since the CD4 cell response to influenza viruses is dominated by Th1 cells, in vitro generated populations of Th2 cells, Th17 cells are investigated, together with adaptive T regulatory cells (aTregs or aTreg cells) as well as Th1 cells and activated CD8 cells, to gain insights into potential roles of CD44 in homeostasis. The present invention provides a survival defect only in Th1 cells. In one aspect, aTreg cells and naturally occurring regulatory T cells (nTregs or nTreg cells) are also resistant to depletion by anti-CD44 in the NOD mouse model (FIG. 8). The failure of memory generation by CD44−/− CD4 cells in the influenza model is due to profound apoptosis during the primary response that engages caspase 8, suggesting death receptor involvement (FIG. 5).

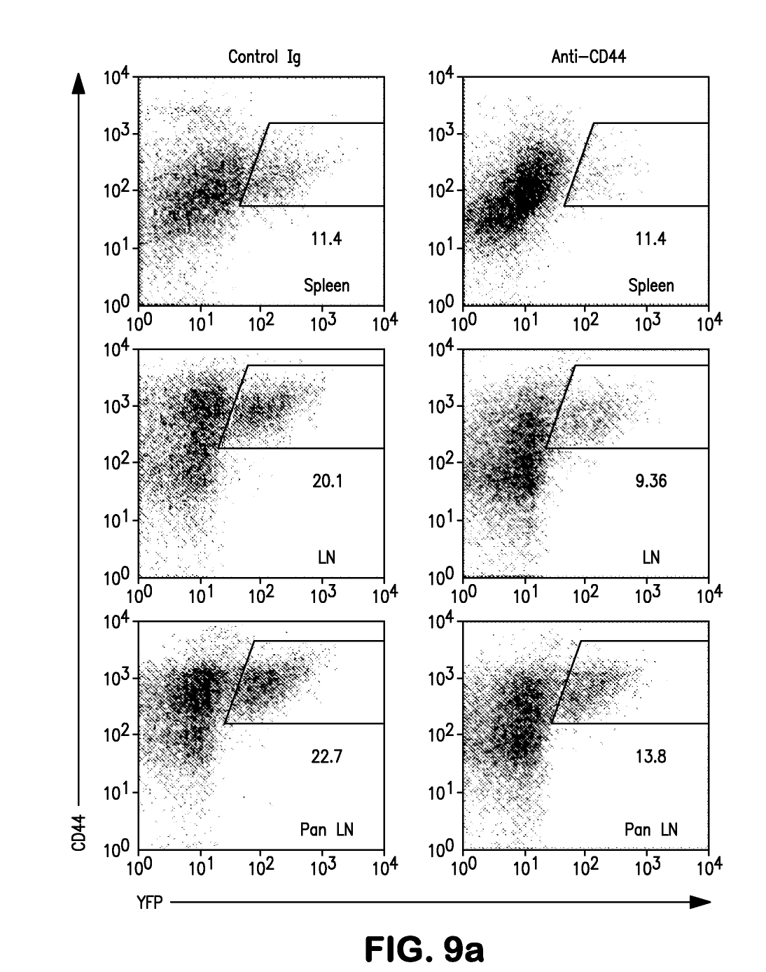

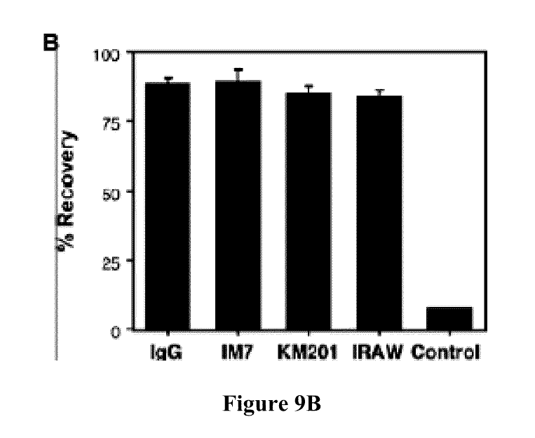

The present invention provides that engagement of CD44 is necessary for Th1 survival via the PI3K/Akt pathway. Blocking HA binding by CD44 in vivo with anti-CD44 throughout the primary response also led to the selective apoptosis of Th1 cells. Since anti-CD44 can prevent development of diabetes in induced models, the present invention provides that Th1 cells can be affected by in vivo treatment with anti-CD44 in NOD mice. To distinguish Th1 cells in situ, IFN-γ reporter mice onto the NOD background are generated (16×). These mice do not develop diabetes at an accelerated rate compared to WT NOD mice. By cell-sorting, all of the IFN-γ-producing CD4 T cells are contained in the YFP+ populational predicted. When prediabetic NOD-YETI mice are treated with anti-CD44, YFP+ cells are lost in all the lymphoid tissues (FIG. 9A). This observation is not due to depletion by cytotoxicity since none of the anti-CD44 mAbs used are cytotoxic (IM7, KM201, IRAW B14)(FIG. 9B). Moreover, CD8+ cells are unaffected. Thus, the present invention provides that anti-CD44 can be used to selectively target Th1 cells in the Type 1 diabetes (T1D) model.

### Example 10

### Targeting CD44 in NOD Mice

To assess effects of targeting CD44 on Th1 cells, Th1 cells are generated from WT or CD44−/− BDC CD4 cells. CD44−/−NOD mice are bred to BDC Ly5.2 NODs to enable a coinjection strategy with WT Thy 1.1+ BDC Th1 cells (FIG. 5) into 6-8 week old WT NOD mice (Thy1.2, Ly5.1) or NOD.Scid mice. The cells are injected in a 1:1 ratio and followed by Annexin V staining and 7AAD uptake to determine if there are detectable differences in the frequency of cells undergoing early apoptosis, for their response by BrdU uptake, and for their survival/recovery at 2-3 day intervals over a 2-week period (d2, 5, 7, 10, 14) from the lymphoid compartment and pancreas. The cells are also injected into separate WT NOD and Scid recipients to test for their capacity to elicit diabetes over 1 month. For a 2nd analysis, WT BDC Ly5.2 Th1 cells are injected into NOD or NOD.Scid recipients and are given anti-CD44 or rat-IgG (300 μg/injection (i.p., 2×/wk) from the time of cell transfer and can be evaluated by the same parameters over the same time frames. To extend these studies to in situ generated Th1 cells, YFP+ CD4 cells from the spleens of prediabetic are sorted and transferred into NOD.Scid recipients (for example recently diagnosed NOD YETI Thy1.1 mice) that are treated with control IgG or anti-CD44 for 4 weeks. Total splenocytes from diabetic NOD YETI mice are used as control since these cells can accelerate T1D in prediabetic NOD mice, and can elicit diabetes in NOD.Scid mice in a 4 week period. For each analysis of the donor cells, 4 mice/group are used with one repeat. For diabetes analysis, 10 mice/group can be used.

To investigate whether anti-CD44 treatment can control hyperglycemia, early onset diabetic NOD mice are treated with anti-CD44 or control IgG at the time of diabetes diagnosis and 2×/week thereafter for up to 8 weeks. Mice that do not show a reduction in BG levels are sacrificed at 4 weeks. Mice in which BG stabilizes and drops are maintained unless BG readings reach 500 mg/dl. Similar studies using NOD YETI Thy1.1 mice can be performed. Mice that recover glucose control are evaluated for pancreatic infiltrates by insulitis index analyses, and in NOD YETI mice, the frequencies of YFP+ CD4+ T cells can be analyzed for comparison, as well as YFP+, CD8+ T cells in the lymphoid compartment and pancreas. 10 mice/group can be used.

To investigate whether anti-CD44 treatment can control reemergence of autoimmunity after islet transplantation, diabetic NOD mice are supported by s.c. implantation of insulin pellets that release 0.1 U/24 hr for ≧30 days to control hyperglycemia. After 2 weeks, ˜200 syngeneic islets are transplanted under the kidney capsule. On the following day, anti-CD44 or control Ig are administered 2×/week, for 1 month. The mice are then evaluated for the presence of infiltrates in the islet grafts. NOD YETI Thy1.1 mice can be used to determine if there are changes in the frequency or distribution of YFP+ CD4 cells or, for comparison, CD8 cells. Similar studies are performed using spleen cells transferred from diabetic NOD YETI Thy1.1 mice into islet transplanted NOD mice as a source of diabetogenic effectors. The present invention provides priming of Th1 cells after islet transplantation by injecting the NOD recipient mice with CFSE-labeled YFP− CD4 cells from BDC NOD YETI Thy1.1 mice that do not receive antibody treatment. Analysis of CFSE dilution by the donor cells in the pancreatic LN, renal LN, superficial LN and spleen and acquisition of YFP. Analysis are performed on days 3, 5, and 7 after cell transfer. At 2 weeks after cell transfer, the grafts for infiltrates by histology (n=4/group) can be evaluated. In mice that do not receive antibody treatment initially after islet transplantation, BG levels for reemergence of diabetes are monitored, and treatment can be performed at the time of relapse to determine if anti-CD44 can be protective as measured by restoration of glucose control when the β-cells are failing.

The present invention provides that targeting CD44 can control development of T1D and anti-CD44 treatment can result in loss of Th1 cells in the NOD model (FIG. 9). The present invention further provides that anti-CD44 treatment can control the reemergence of the autoimmune response as measured by a new T cell response and the appearance of infiltrates in the graft.

### Example 11

### Efficacy of sTregs in Reversing Recent Onset Diabetes

The present invention provides efficacy of aTregs in reversing recent onset diabetes or in protecting syngeneic. The present invention further provides that allogeneic islets after transplantation into recipients with established diabetes can be improved by anti-CD44 treatment.

The present invention provides that in vitro generated FoxP3+ aTregs can reverse and control diabetes indefinitely as long-lasting memory populations (at least 2 years.) The present invention provides the unique regulation by IL-7 for homeostatic maintenance. The data support the feasibility of using such aTregs as a means to reestablish tolerance after diabetes onset since they do not require IL-2 for persistence or maintenance of function as do nTregs. The data support the likelihood that such memory Tregs can be exploited and is an important asset for long-term control of T1D.

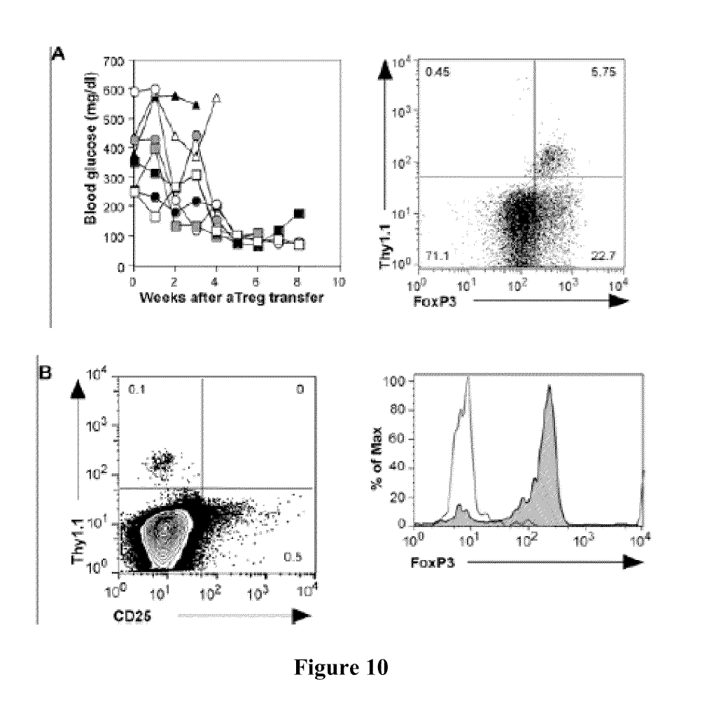

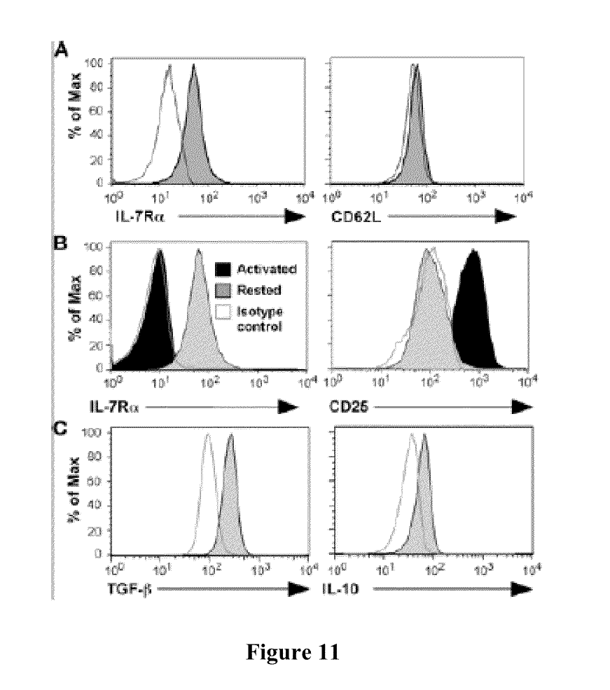

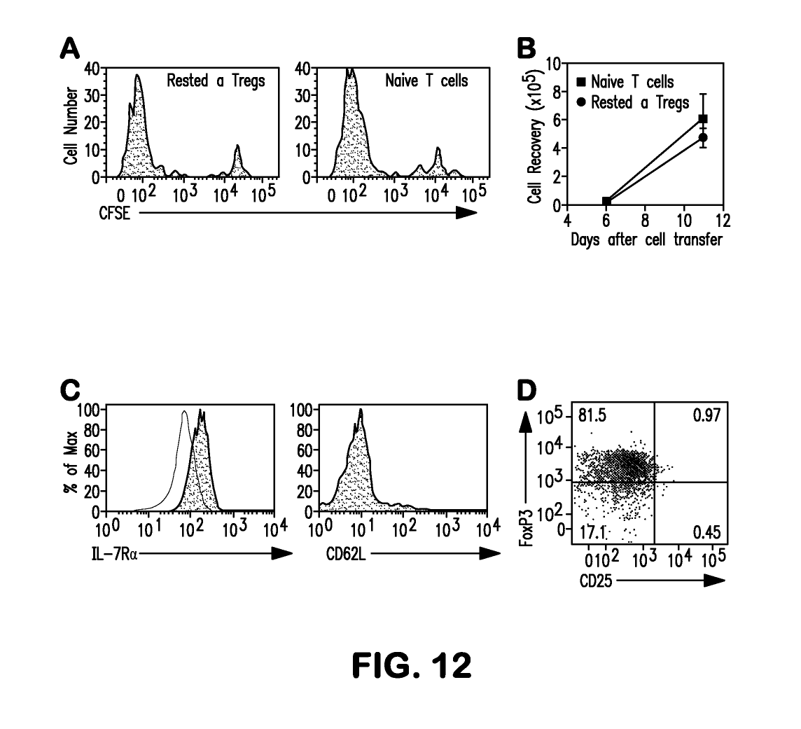

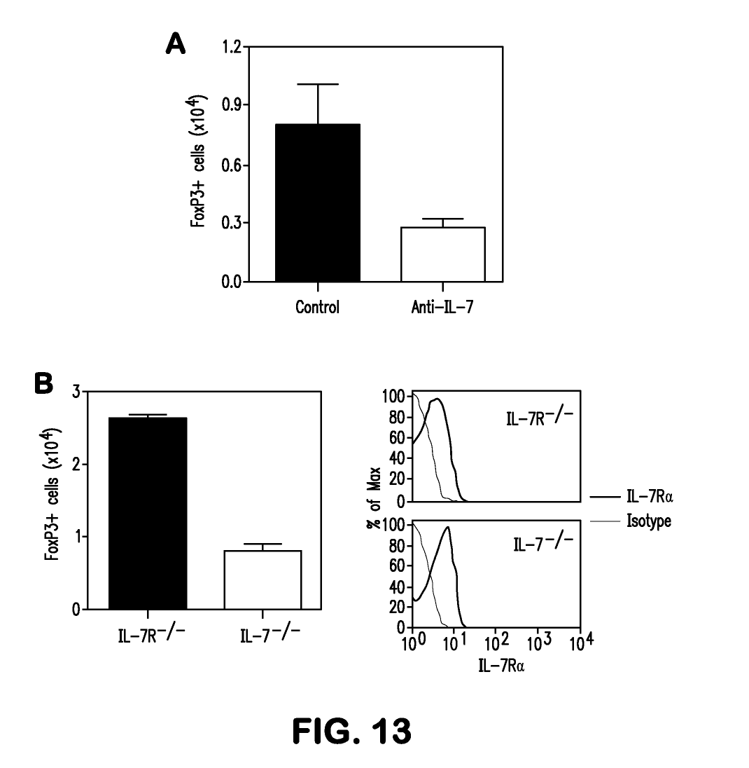

As shown in FIGS. 10A and 10 B, normoglycemia can be reestablished by polyclonal aTreg cells and maintain FoxP3 without CD25. In contrast, where FoxP3 GFP reporter NOD mice is used to distinguish nTreg cells, the majority of nTregs can lose FoxP3 when transferred into mice with established diabetes. There is increasing concern that nTreg are unstable in T1D, at least in part because of a defective response to IL-2. However, aTreg generated can maintain FoxP3 without expression of CD25. Moreover, the cells express high levels of IL-7Rα and low levels of CD62L as memory cells at 9 months after transfer (FIG. 11A). Similar to results for Th1 and Th2 cells, in vitro generated aTreg can up-regulate IL-7Rα and down-regulate CD25 after resting in the absence of stimulation for 3 days (FIG. 11B) and also maintain their capacity to secrete the cytokines TGF-β and IL-10 (FIG. 11C). Unlike nTregs, the cells are phenotypically stable after expansion in lymphopenic (NOD.Scid) recipients (FIG. 12). Expression of IL-7Rα is mirrored by dependence upon IL-7 for survival (FIG. 13), which is normal in T1D patients. aTreg administration can restore normoglycemia in 50-70% of mice if administered at 1 week after diagnosis of diabetes. However, if treatment is delayed until 2 weeks when diabetes is established, reversal of hyperglycemia does not occur and insulin+ β-cells are no longer detected. The data suggest that there is a critical window for reestablishing control of T1D. Since aTregs generated show dependence on Ag for protection, the present invention provides that the efficacy of aTreg cells early after diabetes onset can be improved if the Th1 cell response that orchestrates the loss of β-cells is controlled via anti-CD44-treatment. Since it is critical to determine if anti-CD44 treatment impacts immune responses to infection, the influenza model can be used to determine the effects on T-cell dependent viral clearance in the primary response. The present invention provides that a combination of anti-CD44/aTreg therapy can facilitate the maintenance of β-cells after diabetes is established, but controlled by islet transplantation.

### Example 12

### Effects of Anti-CD44 and aTreg in Protecting Syngeneic and Allogeneic Islets

Since aTregs generated to alloantigens show significant promise in the field of transplantation, the present invention provides that a combination therapy can further enhance protection in this context. Thus, the present invention provides that longevity of syngeneic islet transplants can be improved by anti-CD44 treatment together with transfer of aTregs. Diabetic NOD mice are supported by s.c. implantation of insulin pellets to initially control hyperglycemia. aTregs are generated from FoxP3− (GFP−) NOD BDC Thy 1.1+ CD4 cells and injected one day after insulin supported mice receive islet transplants and treatments with anti-CD44 or control Igare can then be initiated. Further treatments after receiving insulin pellets and islet transplantation are not given to a control group. BG levels are monitored for 8 weeks with antibody treatment, and for a further 8 weeks after treatment are withdrawn. For diabetes analysis, 10 mice/group with one repeat can be used. At the end of the experiment or when mice become diabetic, the distribution of donor cells in the lymphoid compartment and pancreas are analyzed. These experiments are also repeated using polyclonal aTregs generated from FoxP3− (GFP−) NOD Thy 1.1+ CD4 cells.

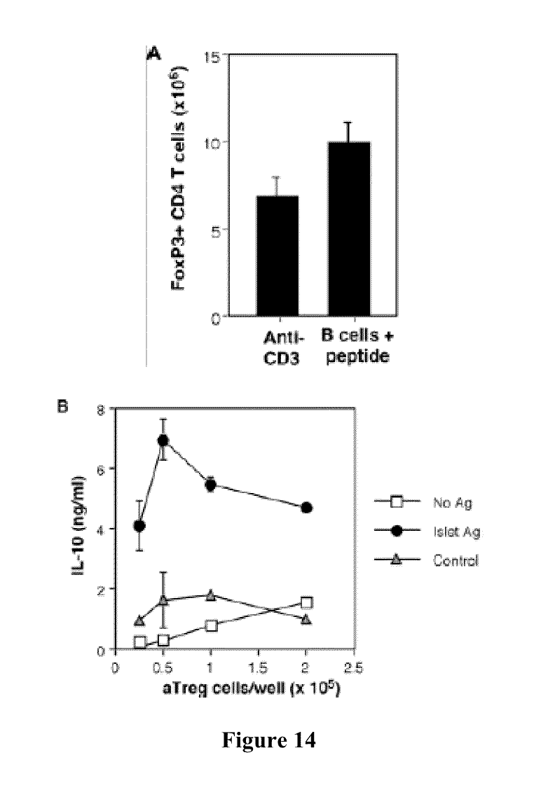

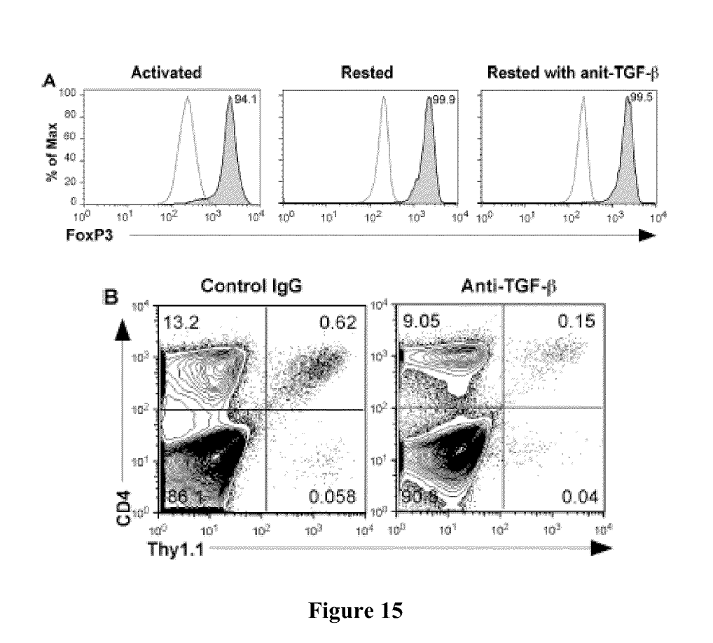

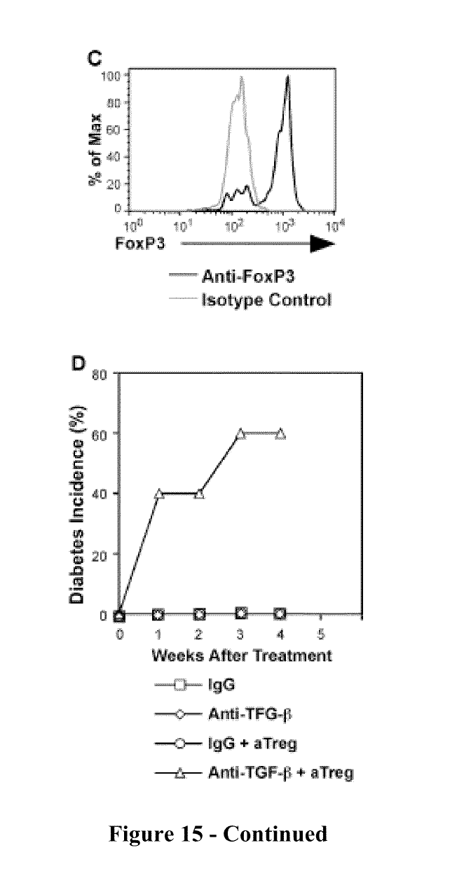

The present invention further provides that aTregs can prevent allogenic islets rejection in vivo. aTregs are generated from FoxP3− (GFP−) NOD Thy1.1+ CD4 cells with allogeneic, or for comparison, syngeneic APC from the T cell-depleted spleen cells from NOD mice (K^{d}, I-Ag⁷, D^{b}), C57BL/6 mice (K^{b}, I-A^{b}D^{b}), or B10. A mice (K^{k}, I-A^{k} D^{k}) in the presence or absence of disrupted islets from mice that are H-2 matched to the APC. Since allogeneic B cells are highly effective inducers of FoxP3 aTregs from naive human CD4 T cells, the present invention provides that splenic B cells can be used to elicit FoxP3+ cells from naive CD4 cells in vitro in the presence of TGF-β (FIG. 14A). The present invention procides that islet proteins can be used to restimulate cytokine secretion by polyclonal aTregs (FIG. 14B). In addition to the aTregs, Trl cells, which effectively prevent allogeneic islet rejection, are generated in the presence of rIL-10. The stability of the aTreg and Trl populations in NOD.Scid recipients are compared to aTregs generated for up-regulation of IL-7Rα with loss of CD25 in vivo (FIGS. 11 and 12). The cells are then injected into diabetic mice supported for 2 weeks by insulin pellets that then receive islets matched to the inducing APC cells with or without anti-CD44 treatment or receive mismatched islets. BG levels are monitored for up to 8 weeks. Control animals without aTreg or anti-CD44 treatment become diabetic after the insulin pellets are exhausted. Rejection of transplanted islets is measured by reemergence of diabetes.

Although the invention has been described with reference to the above example, it will be understood that modifications and variations are encompassed within the spirit and scope of the invention. Accordingly, the invention is limited only by the following claims.

What is claimed is:

1. A method of inhibiting survival of T helper 1 (Th1) memory cells comprising administering to a population of Th1 cells an effective amount of an agent that inhibits CD44 receptor expression or engagement, thereby inhibiting survival of the Th1 cells.

2. The method of claim 1, wherein the Th1 cell comprises a CD4⁺ T cell.

3. The method of claim 1, wherein the agent comprises a CD44 receptor antagonist.

4. The method of claim 1, wherein the agent is a small molecule chemical compound, polypeptide, or nucleic acid molecule.

5. The method of claim 3, wherein the antagonist is an antibody or a binding fragment thereof.

6. The method of claim 1, wherein the administering is performed in vivo, ex vivo, or in vitro.

7. The method of claim 1, wherein the administering is to a subject having an immune system disorder associated with expression or signaling of the CD44 receptor.

8. The method of claim 7, wherein the disorder comprises diabetes mellitus type 1 or an autoimmune disease.

9. The method of claim 7, wherein the disorder is selected from the group consisting of ankylosing spondylitis, Chagas disease, chronic obstructive pulmonary disease, Crohns disease, dermatomyositis, diabetes mellitus type 1, endometriosis, Goodpasture's syndrome, Graves' disease, Guillain-Barré syndrome (GBS), Hashimoto's disease, hidradenitis suppurativa, Kawasaki disease, IgA nephropathy, idiopathic thrombocytopenic purpura, interstitial cystitis, Lupus erythematosus, mixed connective tissue disease, morphea, myasthenia gravis, narcolepsy, neuromyotonia, pemphigus vulgaris, pernicious anaemia, psoriasis, psoriatic arthritis, polymyositis, primary biliary cirrhosis, rheumatoid arthritis, scleroderma, Sjögren's syndrome, stiff person syndrome, temporal arteritis, ulcerative colitis, vasculitis, vitiligo, and Wegener's granulomatosis.

10. The method of claim 8, wherein the method further comprises treating the subject with an islet cell transplantation.

11. The method of claim 10, wherein the islet cells are allogeneic, syngeneic, xenogeneic, or a combination thereof.

12. The method of claim 10, wherein the islet cells are generated ex vivo, in vivo, or in vitro.

13. The method of claim 7, wherein the subject is mammalian.

14. A method of stimulating memory T helper 1 (Th1) cell survival comprising administering to Th1 cells an effective amount of an agent that increases CD44 receptor engagement or expression in the cells, thereby stimulating memory T helper 1 (Th1) cell survival.

15. The method of claim 14, wherein the Th1 cell comprises a CD4⁺ T cell.

16. The method of claim 14, wherein the agent comprises a CD44 receptor agonist.

17. The method of claim 14, wherein the agent comprises glucosaminoglycan hyaluronic acid (HA).

18. The method of claim 14, wherein the administering is to mammalian cells.

19. The method of claim 14, wherein the administering is performed in vivo, ex vivo, or in vitro.

20. The method of claim 14, wherein the administering is in a subject having or at risk of having an infection with a bacterial or viral agent.

21. The method of claim 20, wherein the viral agent is influenza.

22. A method of screening for an agent that modulates memory T helper 1 (Th1) cell survival comprising:
a) administering a test agent to a Th1 cell; and
b) detecting an increase or decrease in CD44 receptor expression or signaling as compared with expression or signaling prior to administering the agent, thereby identifying a test agent as an agent that modulates memory T helper 1 (Th1) cell survival.

23. The method of claim 22, wherein the Th1 cell comprises a CD4⁺ T cell.

24. The method of claim 22, wherein the test agent comprises a CD44 receptor agonist.

25. The method of claim 22, wherein an increase in CD44 receptor expression or signaling is indicative of survival or expansion of the Th1 cells.

26. The method of claim 22, wherein a decrease in CD44 receptor expression or signaling is indicative of lack of survival of the Th1 cells.

27. The method of claim 22, wherein a decrease in CD44 receptor expression is indicative of an inhibition of survival of the Th1 memory cells.

28. The method of claim 22, wherein the administering is performed in vivo, ex vivo, or in vitro.

29. The method of claim 22, wherein the method uses a high throughput format.


SEQUENCE LISTING (2 sequences)

SEQ ID NO: 1 - DNA, 25 bp, Artificial Sequence
  Primer
atcagtcacagacctacccaattcc

SEQ ID NO: 2 - DNA, 26 bp, Artificial Sequence
  Primer
ccaagatgatgagccattctggaatc